SEQUENCE LISTING (32 sequences)

SEQ ID NO: 1 - protein, 331 aa, Homo sapiens
  misc_feature: Human HER3 Protein amino acid sequence
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKAF

SEQ ID NO: 2 - protein, 1342 aa, Homo sapiens
  misc_feature: Human HER3 Protein Precursor amino acid sequence; Full length HER3
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMGGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGGSCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 3 - protein, 11 aa, Artificial Sequence
  Synthetic peptide derived from HER3 protein sequence
NLRVVRGTQVY

SEQ ID NO: 4 - protein, 15 aa, Artificial Sequence
  Synthetic peptide derived from HER3 protein sequence
DKLCHMDTIDWRDIV

SEQ ID NO: 5 - protein, 7 aa, Artificial Sequence
  Synthetic peptide derived from HER3 protein sequence
PCHEVCK

SEQ ID NO: 6 - protein, 15 aa, Artificial Sequence
  Synthetic peptide derived from HER3 protein sequence
ICAPQCNGHCFGPNP

SEQ ID NO: 7 - protein, 7 aa, Artificial Sequence
  Synthetic peptide derived from HER3 protein sequence
NGDPWHK

SEQ ID NO: 8 - protein, 11 aa, Artificial Sequence
  Synthetic peptide derived from HER3 protein sequence
DPLCSSGGCWG

SEQ ID NO: 9 - protein, 11 aa, Artificial Sequence
  Synthetic peptide derived from HER3 protein sequence
CPHGVLGAKGP

SEQ ID NO: 10 - protein, 15 aa, Artificial Sequence
  Synthetic peptide derived from HER3 protein sequence
IAGLVVIFMMLGGTF

SEQ ID NO: 11 - protein, 11 aa, Artificial Sequence
  Synthetic peptide derived from HER3 protein sequence
LERGESIEPLD

SEQ ID NO: 12 - protein, 11 aa, Artificial Sequence
  Synthetic peptide derived from HER3 protein sequence
QLLYSEAKTPI

SEQ ID NO: 13 - protein, 15 aa, Artificial Sequence
  Synthetic peptide derived from HER3 protein sequence
ESIHFGKYTHQSDVW

SEQ ID NO: 14 - protein, 15 aa, Artificial Sequence
  Synthetic peptide derived from HER3 protein sequence
VWELMTFGAEPYAGL

SEQ ID NO: 15 - protein, 8 aa, Artificial Sequence
  Synthetic peptide derived from HER3 protein sequence
ESGPGIAP

SEQ ID NO: 16 - protein, 15 aa, Artificial Sequence
  Synthetic peptide derived from HER3 protein sequence
TLNRPRGSQSLLSPS

SEQ ID NO: 17 - protein, 15 aa, Artificial Sequence
  Synthetic peptide derived from HER3 protein sequence
EAELQEKVSMCRSRS

SEQ ID NO: 18 - protein, 11 aa, Artificial Sequence
  Synthetic peptide derived from HER3 protein sequence
EEDVNGYVMPD

SEQ ID NO: 19 - protein, 7 aa, Artificial Sequence
  Synthetic peptide derived from HER3 protein sequence
MPTAGTT

SEQ ID NO: 20 - protein, 12 aa, Artificial Sequence
  Synthetic peptide derived from HER3 protein sequence
DGGGPGGDYAAM

SEQ ID NO: 21 - protein, 9 aa, Homo sapiens
  misc_feature (1)..(9): HER3 Antigen 1
LAEVPDLLE

SEQ ID NO: 22 - protein, 14 aa, Homo sapiens
  misc_feature (1)..(14): HER3 Antigen 2
YMVMVKCWMIDENI

SEQ ID NO: 23 - protein, 12 aa, Homo sapiens
  misc_feature (1)..(12): HER3 Antigen 3
VMVKCWMIDENI

SEQ ID NO: 24 - protein, 9 aa, Homo sapiens
  misc_feature (1)..(9): HER3 antigen 4
IKVIEDKSG

SEQ ID NO: 25 - protein, 1342 aa, Homo sapiens
  misc_feature (1)..(1342): Human HER3 Protein Precursor amino acid sequence
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 26 - protein, 678 aa, Homo sapiens
  misc_feature (1)..(678): Intracellular domain of HER3  665-1342
YWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 27 - protein, 214 aa, Homo sapiens
  misc_feature (1)..(214): HER3 741-954
IKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENI

SEQ ID NO: 28 - protein, 351 aa, Mus musculus
  misc_feature (1)..(351): C1C2 domains of mouse lactadherin
TEYICQCPVGYSGIHCETGCSTQLGMEGGAIADSQISASSVYMGFMGLQRWGPELARLYRTGIVNAWTASNYDSKPWIQVNLLRKMRVSGVMTQGASRAGRAEYLKTFKVAYSLDGRKFEFIQDESGGDKEFLGNLDNNSLKVNMFNPTLEAQYIKLYPVSCHRGCTLRFELLGCELHGCSEPLGLKNNTIPDSQMSASSSYKTWNLRAFGWYPHLGRLDNQGKINAWTAQSNSAKEWLQVDLGTQRQVTGIITQGARDFGHIQYVASYKVAHSDDGVQWTVYEEQGSSKVFQGNLDNNSHKKNIFEKPFMARYVRVLPVSWHNRITLRLELLGCFEQKLISEEDLNMHTG

SEQ ID NO: 29 - protein, 24 aa, Mus musculus
  misc_feature (1)..(24): leader sequence of mouse lactadherin
MQVSRVLAALCGMLLCASGLFAAS

SEQ ID NO: 30 - protein, 327 aa, Homo sapiens
  misc_feature (1)..(327): C1C2 domains of human lactadherin
YAGNHCETKCVEPLGMENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 31 - protein, 8 aa, Homo sapiens
  misc_feature (1)..(8): leader sequence of human lactadherin
YTCTCLKG

SEQ ID NO: 32 - protein, 664 aa, Homo sapiens
  misc_feature (1)..(664): HER3 extracellular domain and transmembrane domain
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFL

(19) United States
(12) Patent Application Publication
(10) Pub. No.: US 2018/0282736 A1
(43) Pub. Date: Oct. 4, 2018

(54) HER3 VACCINE VECTOR COMPOSITIONS AND METHODS OF USING THE SAME

(71) Applicant: Duke University, Durham, NC (US)

(72) Inventors: Herbert K. Lyerly, Durham, NC (US); Takuya Osada, Durham, NC (US); Zachary C. Hartman, Durham, NC (US)

(73) Assignee: Duke University, Durham, NC (US)

(21) Appl. No.: 15/942,812

(22) Filed: Apr. 2, 2018

Related U.S. Application Data
(60) Provisional application No. 62/479,870, filed on Mar. 31, 2017.
(60) Provisional application No. 62/622,605, filed on Jan. 26, 2018.

(51) Int. Cl. C12N 15/63; A61K 48/00; A61K 45/06; A61K 39/395; A61P 35/00

(52) U.S. Cl. CPC C12N 15/63; A61K 48/0058; A61K 45/06; C12N 2710/10043; A61P 35/00; C12N 2800/24; A61K 39/39558

(57) ABSTRACT

The present inventors provide HER3 vaccine vector compositions and methods of using the same. More specifically, the present inventors demonstrate, in part, that a combination of HER3 vaccine vectors with checkpoint inhibitors suppresses or eliminates cancer cells more effectively than either alone.

### CROSS-REFERENCE TO RELATED PATENT APPLICATIONS

The present application claims the benefit of priority of U.S. Provisional Patent Application No. 62/479,870, filed on Mar. 31, 2017 and U.S. Provisional Patent Application No. 62/622,605, filed on Jan. 26, 2018, the contents of which are incorporated herein by reference in their entireties.

### STATEMENT REGARDING FEDERALLY SPONSORED RESEARCH

This work was supported by grants from the Department of Defense Breast Cancer Research Program Clinical Translational Research Award [W81XWH-12-1-0574 to HKL]. The United States Government has certain rights in this invention.

### SEQUENCE LISTING

This application is being filed electronically via EFS-Web and includes an electronically submitted Sequence Listing in .txt format. The .txt file contains a sequence listing entitled “2018-04-02_5667-00427_ST25.txt” created on Apr. 2, 2018 and is 52,689 bytes in size. The Sequence Listing contained in this .txt file is part of the specification and is hereby incorporated by reference herein in its entirety.

### INTRODUCTION

The human epidermal growth factor receptor (HER) family, consisting of HER1 (also known as EGFR), HER2, HER3 and HER4, drives the progression of many epithelial malignancies. EGFR and HER2 have been extensively studied as mediators of poor prognosis and are credentialed therapeutic targets of both small molecule inhibitors and monoclonal antibody therapy. In contrast, HER3, overexpressed in breast, lung, gastric, head and neck, and ovarian cancers and melanoma, is associated with poor prognosis, but has not been a credentialed therapeutic target because it lacks catalytic kinase activity and is not transforming by itself. However, HER3 is thought to function as a signaling substrate for other HER proteins with which it heterodimerizes (13, 14). Not only are these HER3 heterodimers potent oncogenic signaling drivers, but also they have been described as a cause of therapeutic resistance to anti-EGFR, anti-HER2 and hormonal therapies. Therefore, HER3 is an attractive therapeutic target. Although the lack of a catalytic kinase domain limits direct inhibition with small molecule tyrosine kinase inhibitors (TKIs), HER3 may be targeted with antibodies that either block binding of its ligand neuregulin-1 (NRG-1) (also called heregulin) or cause internalization of HER3, inhibiting downstream signaling. Additionally, the anti-HER2 monoclonal antibody pertuzumab disrupts neuregulin-induced HER2-HER3 dimerization and signaling; however, it is less effective at disrupting the elevated basal state of ligand-independent HER2-HER3 interaction and signaling in HER2-overexpressing tumor cells. There, however, remains a need in the art for therapeutic alternatives to monoclonal antibodies that may target the HER3 protein.

### SUMMARY

In one aspect, the present invention relates to vaccine vectors including a polynucleotide encoding a HER3 polypeptide. The HER3 polypeptide may include a polypeptide having at least 60%, 70%, 80%, 85%, 90%, 92%, 95%, 96%, 97%, 98%, or 99%, or 100% sequence identity to SEQ ID NO: 1 (Human HER3 Protein amino acid sequence), SEQ ID NO: 2 (Human HER3 Protein Precursor amino acid sequence), or any one of SEQ ID NOS: 3-27 or 32 (HER3 Antigenic Epitopes).

In another aspect, compositions including any one of the vaccine vectors described herein and a checkpoint inhibitor are provided.

In a further aspect, pharmaceutical compositions are provided. The pharmaceutical compositions may include a pharmaceutically-acceptable carrier and any one of the vaccine vectors described herein or any one of the combination compositions described herein.

In a still further aspect, methods of treating a cancer or precancer, or of reducing the likelihood of the cancer or precancer developing resistance to a cancer therapeutic or prevention agent in a subject are provided. The methods may include administering a therapeutically effective amount of any one of the combination compositions described herein to the subject having the cancer or precancer. Alternatively, the methods may include administering a therapeutically effective amount of any one of the vaccine vectors described herein to the subject having the cancer or precancer, and administering a therapeutically effective amount of a checkpoint inhibitor. Optionally, each of these methods may further include administering a therapeutically effective amount of the cancer therapeutic or prevention agent to the subject.

### BRIEF DESCRIPTION OF THE DRAWINGS

FIGS. 1A-1D show combined JC-HER3 tumor growth and mouse survival data following treatment with Ad[E1-E2b-]HER3 vaccine. FIG. 1A is a graph showing the antitumor effect after JC-HER3 tumor cells were implanted in HER3-transgenic F1 hybrid mice (5×10⁵ cells/mouse) and mice were immunized on days 3 and 10 with Ad-HER3-FL (SEQ ID NO: 2), Ad[E1-E2b-]GFP (2.6×10E10 vp/injection) or saline. The longitudinal mixed effects model with the maximum likelihood variance estimation method was used to model tumor volume over time. Mean±SE is shown. (Ad-HER3 FL and Ad-GFP: 15 mice/group, saline: 10 mice) *p<0.001 FIG. 1B is a graph showing the effect of Ad[E1-E2b-]HER3-FL vaccine on mouse survival. JC-HER3 tumor cells were implanted in HER3-transgenic F1 hybrid mice and immunized as above in FIG. 1A. Mice were considered censored at the time the tumor volume reached humane endpoint and were euthanized. The Kaplan-Meier method was used to estimate overall survival and treatments were compared using a two-sided log-rank test. FIG. 1C is a blot showing the effect of Ad-HER3 vaccine on HER3 expression by JC-HER3 tumors. When tumor volume reached humane endpoint, mice were sacrificed and tumor tissues were collected. Western blot was performed with anti-hHER3 antibody (Santa Cruz), followed by HRP-conjugated anti-mouse IgG (Cell Signaling) and chemiluminescent development. FIG. 1D is a set of plots showing the effect of Ad-HER3 vaccine on HER3 expression by flow cytometry. JC-HER3 tumors were collected and digested after a vaccine prevention model experiment and pooled by group. hHER3 expression was determined by FACS using PE-anti-hHER3 antibody. Open histograms show HER3 expression, and grey filled histograms show the staining with PE-conjugated isotype control.

FIGS. 2A-2E show analysis of tumor-infiltrating T cells in comparison with splenocytes and lymph node cells. HER3-transgenic mice bearing JC-HER3 tumor and immunized with either Ad-HER3-FL or Ad-GFP were euthanized, and tumors, spleen and lymph nodes were collected from each mouse. Tumors were digested and tumor cells were stained with viability dye and anti-CD3, CD4, CD8, PD-1 and PD-L1 antibodies and analyzed by flow cytometry. FIG. 2A is a graph showing CD3+ T cells as a percentage of total cells in the tumor digest. Percentage of T cells from the tumor of each mouse. Bars show the mean. FIG. 2B is a set of graphs showing CD4 and CD8 T cell population in tumors, spleen, and lymph nodes. Bars represent mean+/−SD percentages of CD4+ and CD8+ cells in CD3+ T cell population for each site. *p<0.05. FIG. 2C is a set of graphs showing CD25+FOXP3 cells in tumor, spleens, and lymph nodes. Bars represent mean+/−SD percentages of CD25+FOXP3+ cells in CD4+ T cell population for each site. Student's T test: *p=0.026 and **p=0.008. FIG. 2D is a set of graphs showing PD-1 expression by T cells in tumors, spleens, lymph nodes and tumors. CD4+ and CD8+ T cells from each site were analyzed for their expression of PD-1 by flow cytometry. Bars represent mean+/−SD for n=3 mice. FIG. 2E is a graph showing PD-L1 expression by tumor cells after Ad-HER3-FL or Ad-GFP vaccination. Expression of PD-L1 by tumor cells was analyzed for each mouse treated with Ad-HER3-FL or Ad-GFP vaccine and shown as percentage. Bars show the mean.

FIG. 3 shows the antitumor effect of Ad-HER3-FL vaccine and PD-1/PD-L1 blockade in HER3 transgenic mice bearing JC-HER3 tumors. Tumor growth inhibition in a prevention model. HER3-transgenic mice were vaccinated with Ad-HER3-FL (2.6×10¹⁰ vp/mouse) on days −11 and −4 and then implanted with JC-HER3 cells (0.5×10⁶ cells/mouse) in the flank on day 0. Mice received intraperitoneal injections of anti-PD-1 or anti-PD-L1 antibody (200 μg/injection) on days 3, 6, 10, 13, 17, and 20. Mean±2SE is shown. *p<0.05, **p<0.01, ***p<0.001

FIGS. 4A-4B show enhanced T cell infiltration into JC-HER3 tumors in mice treated with Ad-HER3-FL vaccine and PD-1/PD-L1 blockade. JC-HER3 tumors from mice immunized with HER3 or control vaccine and treated with/without PD-1/PD-L1 blockade were analyzed for CD3+ T cell infiltration by immunohistochemistry. FIG. 4A is a set of photographs showing increased CD3+ T cell infiltration with Ad-HER3-FL and anti-PD-1 therapy. High power fields were selected randomly at magnification of ×200, and 10 fields that did not include necrotic area were evaluated. Representative high power fields of tumor sections for each group are shown. FIG. 4B is a graph showing the highest CD3+ T cell infiltration was obtained with a combination of Ad-HER3-FL and anti-PD-1 therapy. Two independent observers counted the number of CD3+ T cells in the fields, and the average of 10 fields for each group were shown. Error Bar: SD. *p<0.05, **p<0.01, ***p<0.0001.

FIGS. 5A-5B show immune responses induced by combination of Ad-HER3-FL vaccine and PD-1/PD-L1 blockade. FIG. 5A is a graph showing HER3-specific Cellular Immune Response. HER3-transgenic mice were immunized with Ad-HER3-FL or Ad-GFP and tumor was implanted followed by anti-PD-1 or anti-PD-L1 antibody therapy. At day 25, when mice were euthanized, an IFN-gamma ELISPOT assay was performed with splenocytes from individual mice (n=3 mice per group). HER3 ECD, HER3 ICD and ECD+ICD peptide pool were used as stimulating antigens. Bars represent the number of spots (representing IFN-gamma secreting T cells) +/−SD. P-value: *p<0.05, **p<0.01, ***p<0.001. FIG. 5B is a graph showing the anti-HER3 Humoral Immune Response. When mice were euthanized on day 25, blood was collected from individual mice (n=3 mice per group), and a cell-based ELISA was performed using the serum. Sera from immunized mice were applied at serial dilutions of 1:50 to 1:6400. nIR-conjugated secondary antibody was added at 1:2000 dilution. nIR signals were detected by the LI-COR Odyssey imager at 700 nm channel. The average of difference of nIR signals between the 4T1-HER3 wells and 4T1 wells are shown.

FIG. 6 shows tumor growth inhibition improved with sequential Ad-HER3 vaccination followed by immune checkpoint blockade. HER3 transgenic mice were implanted with JC-HER3 cells (0.5×10⁶ cells/mouse) in the flank on day 0, then vaccinated with Ad-HER3-FL or control Ad-GFP (2.6×10¹⁰ vp/mouse) on days 3 and 10. Mice received intraperitoneal injection of anti-PD-L1 antibody and/or anti-CTLA4 antibody or control IgG (200 μg/injection) twice a week (on days 3, 7, 10, 14, 17 and 21). Mean±2SE is shown. *p<0.005, **p<0.001

FIGS. 7A-7B show immune responses induced by combination of Ad-HER3-FL vaccine and either PD-1/PD-L1 blockade or CTLA4 blockade. FIG. 7A is a graph showing anti-HER3 Cellular Immune Response: IFN-gamma ELISPOT assay was performed using splenocytes collected at the euthanasia of mice. HER3 ECD (SEQ ID NO: 32), HER3 ICD (SEQ ID NO: 26) or mixture of ECD and ICD peptide pool (SEQ ID NOs: 3-27) were used as stimulating antigens. HIV peptide pool was used as negative control. Numbers of spots in medium alone (no stimulating antigen) were subtracted and shown. Error bars: SD. *p<0.05, **p<0.01, ***p<0.005. FIG. 7B is a graph showing anti-HER3 Humoral Immune Response: Cell-base ELISA for anti-HER3 antibody was performed using mouse serum collected at the euthanasia of mice. Titrated mouse sera were added to 4T1 cell-coated 96-well plate or 4T1-HER3 cell-coated 96-well plate. After incubation, nIR-conjugated anti-mouse IgG antibody was added. nIR signals were detected by the LI-COR Odyssey imager at 700 nm channel. The average of difference of nIR signals between the 4T1-HER3 wells and 4T1 wells are shown.

FIGS. 8A-8D show the effect of Ad-HER3-FL vaccine and checkpoint inhibitors on T cell subpopulations in Spleens and Tumors of vaccinated mice. FIG. 8A is a graph showing tumor infiltrating lymphocytes (TIL) were isolated from tumor tissues. Percentages of CD25+ Foxp3+ in CD4 T cells in TILs. FIG. 8B is a graph showing the spleens T cell numbers after spleens were harvested and analyzed by flow cytometry assay. Percentages of CD25+ Foxp3+ in CD4 T cells in splenocytes. FIG. 8C is a graph showing the CD8/Treg ratio in Tumor infiltrating lymphocytes (TIL). FIG. 8D is a graph showing the CD8/Treg ratio in splenocytes. *p<0.05, **p<0.01, ***p<0.001.

FIGS. 9A-9B show anti-HER3 immune response induced by Ad-HER3-FL vaccination in human HER3-transgenic mice. FIG. 9A is a graph showing the cellular immune response. Human HER3-transgenic mice were vaccinated at 0 and 14 days with Ad-HER3-FL, control Ad-GFP (2.6×10¹⁰ vp/vaccination), or saline. The mice were sacrificed on day 21 and splenocytes were harvested. IFN-g ELISPOT assays were performed with splenocytes using peptide pools derived from HER3-ECD, HER3-ICD, or HIV (negative control) as stimulating antigens. The number of spots indicating T cells secreting IFN-g in response to the respective peptide pools is reported. Average values of 4 mice from each group are shown. P-value: *p<0.0001. FIG. 9B is a graph showing the humoral immune response. 4T1 and 4T1-HER3 cells seeded into 96 well flat-bottomed plates the day prior were incubated for 1 h on ice with serum (at serial dilutions of 1:50 to 1:6400) from 4 mice vaccinated as in FIG. 9A and collected at the time of sacrifice. The cells were then fixed with 1% formaldehyde and HRP-labeled Goat anti-mouse IgG (1:2000) was added. After 1 h incubation, TMB was added for 5 min for color development and H₂SO₄ was added to stop the reaction. The average differences of OD450 values ([value for 4T1-HER3]−[value for 4T1]) are shown.

### DETAILED DESCRIPTION

The present inventors hypothesized that activation of T cells by a vaccine against a tumor antigen would lead to increased tumor infiltration of antigen-specific T cells and the anti-tumor activity of these T cells would be enhanced by checkpoint blockade.

In order to activate immune responses against HER3, the present inventors, in the non-limiting Examples, generated a recombinant adenoviral vector expressing full length human HER3 (SEQ ID NO: 2; Ad-HER3-FL) and demonstrated that it elicited HER3-specific humoral and cellular immune responses in HER3-transgenic mice, thus breaking tolerance. They also developed breast cancer models expressing HER3 and surprisingly demonstrated that delayed tumor progression with preventive and therapeutic vaccination was associated with an accumulation of PD-1 expressing-tumor infiltrating lymphocytes (TIL). A combination of the Ad-HER3 vaccine with either anti-PD-1 or anti-PD-L1 antibodies suppressed or eliminated HER3-expressing breast cancer more effectively than either alone when used in preventive models, but had only a modest anti-tumor effect in therapeutic models. A combination of anti-CTLA4 and Ad-HER3 vaccine demonstrated a greater anti-tumor effect in the therapeutic model.

Expression of human epidermal growth factor family member 3 (HER3), a critical heterodimerization partner with EGFR and HER2, promotes more aggressive biology in breast and other epithelial malignancies. As such, inhibiting HER3 could have broad applicability to the treatment of EGFR- and HER2-driven tumors. Although lack of a functional kinase domain limits use of receptor tyrosine kinase inhibitors, HER3 contains antigenic targets for T cells and antibodies. Using novel human HER3 transgenic mouse models of breast cancer, the present inventors demonstrate that immunization with recombinant adenoviral vectors encoding full length human HER3 (Ad-HER3-FL) induces HER3-specific T cells and antibodies, alters the T cell infiltrate in tumors, and influences responses to immune checkpoint inhibitions. Both preventative and therapeutic Ad-HER3-FL immunization delayed tumor growth, but were associated with both intratumoral PD-1 expressing CD8+ T cells and regulatory CD4+T cell infiltrates. Immune checkpoint inhibition with either anti-PD-1, anti-PD-L1 antibodies increased intratumoral CD8+ T cell infiltration and eliminated tumor following preventive vaccination with Ad-HER3-FL vaccine. The combination of dual PD-1/PD-L1 and CTLA4 blockade slowed the growth of tumor in response to Ad-HER3-FL in the therapeutic model. The present inventors conclude that HER3-targeting vaccines activate HER3-specific T cells and induce anti-HER3 specific antibodies, which alters the intratumoral T cell infiltrate and responses to immune checkpoint inhibition.

### ABBREVIATIONS

The following abbreviations are used throughout this specification:
Ad Adenovirus
CTLA4 Cytotoxic T-Lymphocyte-Associated Protein 4
ECD Extracellular domain
EGFR Epidermal Growth Factor Receptor
ELISA Enzyme-Linked Immunosorbent Assay
ELISPOT Enzyme-Linked ImmunoSpot
FL Full length
HER3 Human Epidermal Growth Factor Receptor 3
HER2 Human Epidermal Growth Factor Receptor 2
ICD Intracellular domain
IHC Immunohistochemistry
OS Overall survival
PD-1 Programmed Death Receptor 1
PD-L1 Programmed Death Receptor Ligand 1
TIL Tumor infiltrating lymphocytes

### Vaccine Vectors

In one aspect, the present invention relates to vaccine vectors including a polynucleotide encoding a HER3 polypeptide. The HER3 polypeptide may include a polypeptide having at least 60%, 70%, 80%, 85%, 90%, 92%, 95%, 96%, 97%, 98%, or 99%, or 100% sequence identity to SEQ ID NO: 1 (Human HER3 Protein amino acid sequence), SEQ ID NO: 2 (Human HER3 Protein Precursor amino acid sequence), or any one of SEQ ID NOS: 3-27, 32 (HER3 Antigenic Epitopes).

As used herein, the terms “protein” or “polypeptide” or “peptide” may be used interchangeably to refer to a polymer of amino acids. A “polypeptide” as contemplated herein typically comprises a polymer of naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins contemplated herein may be further modified in vitro or in vivo to include non-amino acid moieties.

The HER3 polypeptides disclosed herein may include “variant” HER3 polypeptides. As used herein the term “wild-type” is a term of the art understood by skilled persons and means the typical form of an organism, strain, gene or characteristic as it occurs in nature as distinguished from variant forms. As used herein, a “variant, “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a HER3 variant molecule may have one or more insertions, deletions, or substitution of at least one amino acid residue relative to the HER3 “wild-type” polypeptide sequence of a particular organism. The polypeptide sequences of the “wild-type” HER3 polypeptides from, for example, humans are presented as SEQ ID NOS: 1-27 and 32. The full length HER3 polypeptide is presented as SEQ ID NO: 1 or 2. These sequences may be used as reference sequences.

The HER3 polypeptides provided herein may be full-length polypeptides (as in SEQ ID NOS: 1 or 2) or may be fragments of the full-length polypeptide (e.g., SEQ ID NO: 3-27 or 32). The HER3 polypeptides may be encompassed in a fragment of full-length HER3. For example, the HER3 polypeptides are all within the intracellular domain of HER3 which is presented as SEQ ID NO: 26. As used herein, a “fragment” is a portion of an amino acid sequence which is identical in sequence to but shorter in length than a reference sequence. A fragment may comprise or consist of up to the entire length of the reference sequence (e.g., SEQ ID NOS: 1-27, 32), minus at least one amino acid residue. In some embodiments, a fragment of the HER3 polypeptides may comprise or consist of at least 5, 6, 7, 8, 9, or more amino acids thereof. Preferably, a fragment of a HER3 antigenic polypeptide includes the amino acid residues responsible for eliciting an immune response such as a T cell response in a subject.

The vaccine vectors may include a promoter operably connected to the polynucleotide encoding any one of the HER3 polypeptides described herein. The vectors may include an origin of replication suitable to allow maintenance of the polynucleotide within a prokaryotic or eukaryotic host cell or within a viral nucleic acid. The vector may be viral vectors including, without limitation, an adenovirus, adeno-associated virus, fowlpox, vaccinia, viral equine encephalitis virus, or venezuelan equine encephalitis virus. In some embodiments, the vector is a DNA-based plasmid vector or DNA vaccine vector.

In some embodiments, the vaccine vector may include an adenovirus serotype 5 vector with E2b, E1, and E3 genes deleted.

The vaccine vector may also be mini-circle DNA (mcDNA) vectors. Mini-circle DNA vectors are episomal DNA vectors that are produced as circular expression cassettes devoid of any bacterial plasmid DNA backbone. See, e.g. System Biosciences, Mountain View CA, MN501A-1. Their smaller molecular size enables more efficient transfections and offers sustained expression over a period of weeks as compared to standard plasmid vectors that only work for a few days. The minicircle constructs can be derived from a plasmid with a bacterial origin of replication and optionally antibiotic resistance genes flanked by att sites to allow for recombination and exclusion of the DNA between the att sites and formation of the minicircle DNA.

As used herein, a “heterologous promoter” refers to any promoter not naturally associated with a polynucleotide to which it is operably connected. Promoters useful in the practice of the present invention include, without limitation, constitutive, inducible, temporally-regulated, developmentally regulated, chemically regulated, physically regulated (e.g., light regulated or temperature-regulated), tissue-preferred, and tissue-specific promoters. Promoters may include pol I, pol II, or pol III promoters. In mammalian cells, typical promoters include, without limitation, promoters for Rous sarcoma virus (RSV), human immunodeficiency virus (HIV-1), cytomegalovirus (CMV), SV40 virus, and the like as well as the translational elongation factor EF-la promoter or ubiquitin promoter. Those of skill in the art are familiar with a wide variety of additional promoters for use in various cell types.

Suitably the polynucleotide encodes the full-length HER3 antigenic polypeptide, however, polynucleotides encoding partial, fragment, mutant, variant, or derivative HER3 antigenic polypeptide are also provided. In some embodiments, the polynucleotides may be codon-optimized for expression in a particular cell.

The polynucleotide encoding any of the HER3 polypeptides described herein may also be fused in frame to a second polynucleotide encoding fusion partners such as fusion polynucleotides or polypeptides which provide additional functionality to the antigenic cargo. For example, the second polynucleotide may encode a polypeptide that would target the HER3 polypeptide to the exosome, or would enhance presentation of the HER3 polypeptide, or would stimulate immune responses to the HER3 polypeptide. In some embodiments, the vaccine vectors described herein include a polynucleotide encoding any of the HER3 polypeptides described herein that is fused in frame to a second polynucleotide encoding a lactadherin polypeptide or portions thereof. Lactadherin is a protein that is trafficked to exosomes though its C1C2 domain, a lipid binding domain. The lactadherin polypeptide may include SEQ ID NOS: 28-31 or a homolog thereof.

### Combination Compositions

In another aspect, compositions including any one of the vaccine vectors described herein and a checkpoint inhibitor or a polynucleotide encoding a checkpoint inhibitor are provided.

As used herein, a “checkpoint inhibitor” is an agent, such as antibody or small molecule, which blocks the immune checkpoint pathways in immune cells that are responsible for maintaining self-tolerance and modulating the degree of an immune response. Exemplary checkpoint inhibitors include, without limitation, antibodies or other agents targeting programmed cell death protein 1 (PD1, also known as CD279), programmed cell death 1 ligand 1 (PD-L1, also known as CD274), PD-L2, cytotoxic T-lymphocyte antigen 4 (CTLA4, also known as CD152), A2AR, CD27, CD28, CD40, CD80, CD86, CD122, CD137, OX40, GITR, ICOS, TIM-3, LAG3, B7-H3, B7-H4, BTLA, IDO, KIR, or VISTA. Suitable anti-PD1 antibodies include, without limitation, lambrolizumab (Merck MK-3475), nivolumab (Bristol-Myers Squibb BMS-936558), AMP-224 (Merck), and pidilizumab (CureTech CT-011). Suitable anti-PD-L1 antibodies include, without limitation, MDX-1105 (Medarex), MEDI4736 (Medimmune) MPDL3280A (Genentech/Roche) and BMS-936559 (Bristol-Myers Squibb). Exemplary anti-CTLA4 antibodies include, without limitation, ipilimumab (Bristol-Myers Squibb) and tremelimumab (Pfizer).

In some embodiments, the checkpoint inhibitor may be selected from the group consisting of an anti-PD-1 agent, an anti-PDL1 agent, and an anti-CTLA-4 agent.

In some embodiments, the checkpoint inhibitor may be the form of a polynucleotide encoding a checkpoint inhibitor. For example, with regards to antibody-based checkpoint inhibitors, the checkpoint inhibitor may be in the form of a DNA polynucleotide that is included in any one of the vaccine vectors disclosed herein or may be a DNA polynucleotide that is included in a different expression vector or plasmid. Alternatively, the checkpoint inhibitor may be in the form of a RNA polynucleotide such as, without limitation, an mRNA.

The combination compositions described herein may also include two checkpoint inhibitors, wherein one checkpoint inhibitor comprises an anti-PD-1 agent or an anti-PDL1 agent and the other checkpoint inhibitor comprises an anti-CTLA-4 agent.

The combination compositions may further include a cancer therapeutic or prevention agent. As used herein, a “cancer therapeutic or prevention agent” may be any agent capable of treating the cancer or inhibiting growth of cancer cells. Suitable agents include those which target HER2, HER1/EGFR, estrogen receptor or IGF1R. The cancer therapeutic or prevention agent may be trastuzumab, lapatinib, pertuzumab or another HER2 targeting therapeutic agent or it may be an EGFR targeting therapeutic agent such as cetuximab or erlotanib, or it may be an antiestrogen, or an agent that prevents estrogen synthesis such as an aromatase inhibitor.

### Pharmaceutical Compositions

In a further aspect, pharmaceutical compositions are provided. The pharmaceutical compositions may include a pharmaceutically-acceptable carrier and any one of the vaccine vectors described herein or any one of the combination compositions described herein.

The pharmaceutical compositions may include a pharmaceutical carrier, excipient, or diluent, which are nontoxic to the cell or subject being exposed thereto at the dosages and concentrations employed. Often a pharmaceutical diluent is in an aqueous pH buffered solution. Examples of pharmaceutical carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ brand surfactant, polyethylene glycol (PEG), and PLURONICS™ surfactant.

The pharmaceutical compositions may include adjuvants to increase immunogenicity of the composition. In some embodiments, these pharmaceutical compositions comprise one or more of a mineral adjuvant, gel-based adjuvant, tensoactive agent, bacterial product, oil emulsion, particulated adjuvant, fusion protein, and lipopeptide. Mineral salt adjuvants include aluminum adjuvants, salts of calcium (e.g. calcium phosphate), iron and zirconium. Gel-based adjuvants include aluminum gel-based adjuvants and acemannan. Tensoactive agents include Quil A, saponin derived from an aqueous extract from the bark of Quillaja saponaria; saponins, tensoactive glycosides containing a hydrophobic nucleus of triterpenoid structure with carbohydrate chains linked to the nucleus, and QS-21. Bacterial products include cell wall peptidoglycan or lipopolysaccharide of Gram-negative bacteria (e.g. from Mycobacterium spp., Corynebacterium parvum, C. granulosum, Bordetella pertussis and Neisseria meningitidis), N-acetyl muramyl-L-alanyl-D-isoglutamine (MDP), different compounds derived from MDP (e.g. threonyl-MDP), lipopolysaccharides (LPS) (e.g. from the cell wall of Gram-negative bacteria), trehalose dimycolate (TDM), cholera toxin or other bacterial toxins, and DNA containing CpG motifs. Oil emulsions include FIA, Montanide, Adjuvant 65, Lipovant, the montanide family of oil-based adjuvants, and various liposomes. Among particulated and polymeric systems, poly (DL-lactide-coglycolide) microspheres have been extensively studied and find use herein. Notably, several of the delivery particles noted above may also act as adjuvants.

In some embodiments, the pharmaceutical compositions further include cytokines (e.g. IFN-γ, granulocyte-macrophage colony stimulating factor (GM-CSF) IL-2, or IL-12) or immunostimulatory molecules such as FasL, CD40 ligand or a toll-like receptor agonist, or carbohydrate adjuvants (e.g. inulin-derived adjuvants, such as, gamma inulin, algammulin, and polysaccharides based on glucose and mannose, such as glucans, dextrans, lentinans, glucomannans and galactomannans). In some embodiments, adjuvant formulations are useful in the present invention and include alum salts in combination with other adjuvants such as Lipid A, algammulin, immunostimulatory complexes (ISCOMS), which are virus like particles of 30-40 nm and dodecahedric structure, composed of Quil A, lipids, and cholesterol.

In some embodiments, the additional adjuvants are described in Jennings et al. Adjuvants and Delivery Systems for Viral Vaccines-Mechanisms and Potential. In: Brown F, Haaheim L R, (eds). Modulation of the Immune Response to Vaccine Antigens. Dev. Biol. Stand, Vol. 92. Basel: Karger 1998; 19-28 and/or Sayers et al. J Biomed Biotechnol. 2012; 2012: 831486, and/or Petrovsky and Aguilar, Immunology and Cell Biology (2004) 82,488-496.

In some embodiments, the adjuvant is an aluminum gel or salt, such as aluminum hydroxide, aluminum phosphate, and potassium aluminum sulfate, AS04 (which is composed of aluminum salt and MPL), and ALHYDROGEL. In some embodiments, the aluminum gel or salt is a formulation or mixture with any of the additional adjuvants described herein.

In some embodiments, pharmaceutical compositions include oil-in-water emulsion formulations, saponin adjuvants, ovalbumin, Freunds Adjuvant, cytokines, and/or chitosans. Illustrative compositions comprise one or more of the following.

(1) ovalbumin (e.g. ENDOFIT);

(2) oil-in-water emulsion formulations, with or without other specific immunostimulating agents, such as: (a) MF59 (PCT Publ. No. WO 90/14837), which may contain 5% Squalene, 0.5% Tween 80, and 0.5% Span 85 (optionally containing various amounts of MTP-PE) formulated into submicron particles, (b) SAF, containing 10% Squalane, 0.4% Tween 80, 5% pluronic-blocked polymer L121, and thr-MDP either microfluidized into a submicron emulsion or vortexed to generate a larger particle size emulsion, (c) RIBI adjuvant system (RAS), (RIBI IMMUNOCHEM, Hamilton, Mo.) containing 2% Squalene, 0.2% Tween 80, and, optionally, one or more bacterial cell wall components from the group of monophosphorylipid A (MPL), trehalose dimycolate (TDM), and cell wall skeleton (CWS), including MPL+CWS (DETOX™); and (d) ADDAVAX (Invitrogen);

(3) saponin adjuvants, such as STIMULON (Cambridge Bioscience, Worcester, Mass.);

(4) Complete Freunds Adjuvant (CFA) and Incomplete Freunds Adjuvant (IFA);

(5) cytokines, such as interleukins (by way of non-limiting example, IL-1, IL-2, IL-4, IL-5, IL-6, IL-7, IL-12, etc.), interferons (e.g., gamma interferon), macrophage colony stimulating factor (M-CSF), tumor necrosis factor (TNF), etc;

(6) chitosans and other derivatives of chitin or poly-N-acetyl-D-glucosamine in which the greater proportion of the N-acetyl groups have been removed through hydrolysis; and

(7) other substances that act as immunostimulating agents to enhance the effectiveness of the composition, e.g., monophosphoryl lipid A.

In other embodiments, adjuvants include a flagellin-based agent, an aluminium salt or gel, a pattern recognition receptors (PRR) agonist, CpG ODNs and imidazoquinolines. In some embodiments, adjuvants include a TLR agonist (e.g. TLR1, and/or TLR2, and/or TLR3, and/or TLR4, and/or TLR5, and/or TLR6, and/or TLR7, and/or TLR8, and/or TLR9, and/or TLR10, and/or TLR11, and/or TLR12, and/or TLR13), a nucleotide-binding oligomerization domain (NOD) agonist, a stimulator of interferon genes (STING) ligand, or related agent.

### Methods

In a still further aspect, methods of treating a cancer or precancer, or of reducing the likelihood of the cancer or precancer developing resistance to a cancer therapeutic or prevention agent in a subject are provided. The methods may include administering a therapeutically effective amount of any one of the combination compositions described herein to the subject having the cancer or precancer. Alternatively, the methods may include administering a therapeutically effective amount of any one of the vaccine vectors described herein to the subject having the cancer or precancer, and administering a therapeutically effective amount of a checkpoint inhibitor or a polynucleotide encoding a checkpoint inhibitor. Optionally, each of these methods may further include administering a therapeutically effective amount of the cancer therapeutic or prevention agent to the subject.

In some embodiments of the present methods, two checkpoint inhibitors may be administered wherein one checkpoint inhibitor comprises an anti-PD-1 agent or an anti-PDL1 agent and the other checkpoint inhibitor comprises an anti-CTLA-4 agent.

In some embodiments, the administration of the vaccine vector and the checkpoint inhibitor results in decreased tumor growth rate or decreased tumor size after administration as compared to administration of either the vaccine vector or checkpoint inhibitor alone.

The subject may be any mammal, suitably a human, domesticated animal such as a dog or cat, or a mouse or rat.

Exemplary cancers in accordance with the present invention include, without limitation, primary and metastatic breast, ovarian, liver, pancreatic, prostate, bladder, lung, osteosarcoma, pancreatic, gastric, esophageal, colon, skin cancers (basal and squamous carcinoma; melanoma), testicular, colorectal, urothelial, renal cell, hepatocellular, leukemia, lymphoma, multiple myeloma, head and neck, and central nervous system cancers or pre-cancers. In some embodiments, the cancer may be HER2 positive. The cancer may be selected from any cancer capable of developing resistance to a therapeutic agent by increasing expression or activation of a protein by the cancer cells. In particular the cancer may be any cancer capable of developing resistance to a therapeutic agent which targets a HER family tyrosine kinase, suitably HER2 or EGFR or the estrogen receptor, suitably anti-estrogens. The cancer may develop resistance by increasing the expression of HER3, which although not a kinase, will dimerize with another HER family kinase and allow for signaling to occur.

Thus the HER3 vaccine vectors provided herein may be administered in combination with other therapeutic agents including those targeting a HER family kinase such a s HER2 or EGFR such as a tyrosine kinase inhibitor or may be combined with a checkpoint inhibitor or may be combined with both a HER targeting agent and a checkpoint inhibitor. The vaccines need not be administered at the same time as the other agents. The HER3 vaccine vectors may be administered before, at the same time or after the other agents. In addition to the HER3 vaccine vectors provided herein, other vaccine vectors may also be used such as those in published applications WO 2016/007499; WO 2016/007504; and WO 2017/120576. Each of these vaccine vectors may be combined with at least one checkpoint inhibitor.

Treating cancer includes, but is not limited to, reducing the number of cancer cells or the size of a tumor in the subject, reducing progression of a cancer to a more aggressive form, reducing proliferation of cancer cells or reducing the speed of tumor growth, killing of cancer cells, reducing metastasis of cancer cells or reducing the likelihood of recurrence of a cancer in a subject. Treating a subject as used herein refers to any type of treatment that imparts a benefit to a subject afflicted with a disease or at risk of developing the disease, including improvement in the condition of the subject (e.g., in one or more symptoms), delay in the progression of the disease, delay the onset of symptoms or slow the progression of symptoms, etc.

Co-administration of one or more checkpoint inhibitors or other cancer therapeutic or prevention agent with the HER3 vaccine vector may be administered in any order, at the same time or as part of a unitary composition. The compositions and combinations may be administered such that one agent is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks or more.

In some embodiments, the vaccine vector is administered prior to or simultaneously with the checkpoint inhibitor.

In some embodiments, the vaccine vector is administered prior to the administration of the optional cancer therapeutic or prevention agent.

An “effective amount” or a “therapeutically effective amount” as used herein means the amount of a composition that, when administered to a subject for treating a state, disorder or condition is sufficient to effect a treatment (as defined above). The therapeutically effective amount will vary depending on the composition, formulation or combination, the disease and its severity and the age, weight, physical condition and responsiveness of the subject to be treated.

The compositions (i.e., those including the vaccine vector(s), checkpoint inhibitor(s), or cancer therapeutic or prevention agent(s)) described herein may be administered by any means known to those skilled in the art, including, but not limited to, oral, topical, intranasal, intraperitoneal, parenteral, intravenous, intramuscular, subcutaneous, intrathecal, transcutaneous, nasopharyngeal, or transmucosal absorption. Thus the compositions may be formulated as an ingestable, injectable, topical or suppository formulation. The composition may also be delivered with in a liposomal or time-release vehicle. Administration of the compositions to a subject in accordance with the invention appears to exhibit beneficial effects in a dose-dependent manner. Thus, within broad limits, administration of larger quantities of the compositions is expected to achieve increased beneficial biological effects than administration of a smaller amount. Moreover, efficacy is also contemplated at dosages below the level at which toxicity is seen.

It will be appreciated that the specific dosage administered in any given case will be adjusted in accordance with the compositions being administered, the disease to be treated or inhibited, the condition of the subject, and other relevant medical factors that may modify the activity of the compositions or the response of the subject, as is well known by those skilled in the art. For example, the specific dose for a particular subject depends on age, body weight, general state of health, diet, the timing and mode of administration, the rate of excretion, medicaments used in combination and the severity of the particular disorder to which the therapy is applied. Dosages for a given patient can be determined using conventional considerations, e.g., by customary comparison of the differential activities of the compound of the invention and of a known agent such as tocopherol, such as by means of an appropriate conventional pharmacological or prophylactic protocol.

The maximal dosage for a subject is the highest dosage that does not cause undesirable or intolerable side effects. The number of variables in regard to an individual prophylactic or treatment regimen is large, and a considerable range of doses is expected. The route of administration will also impact the dosage requirements. It is anticipated that dosages of the compound will reduce symptoms of the condition at least 10%, 20%, 30%, 40%, 50%, 60%, 70%, 80%, 90% or 100% compared to pre-treatment symptoms or symptoms is left untreated. It is specifically contemplated that pharmaceutical preparations and compositions may palliate or alleviate symptoms of the disease without providing a cure, or, in some embodiments, may be used to cure the disease or disorder.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1—Vaccination Targeting Human HER3 Alters the Phenotype of Infiltrating T Cells and Responses to Immune Checkpoint Inhibition

### Results

### Adenoviral Vectors Encoding HER3 Elicit Anti-HER3 T Cell and Antibody Responses in HER3-Transgenic Mice

In order to develop a potent, clinically relevant vaccine to induce HER3 specific T and B cell responses, we modified the well-characterized first generation adenovirus serotype 5 vector Ad5[E1-E3-] by inserting the gene for full length human HER3 to generate a viral vector construct referred to subsequently as Ad[E1-]HER3. A recognized challenge with first generation adenoviral vectors is that pre-existing or induced neutralizing antibodies reduce their immunogenicity. Because we have previously demonstrated potent immunogenicity despite anti-vector neutralizing antibodies by using recombinant adenovirus serotype 5 vectors deleted of the early gene E2b in addition to the deletion of E1 and E3 genes (Ad5[E1-E2b-])(29), we generated an Ad5[E1-E2b-] vector expressing full length human HER3 (Ad-HER3-FL). To test the immunogenicity of this HER3 vaccine in the stringent setting where human HER3 is a self-antigen, we first developed a human HER3-transgenic mouse. Further, we crossed the human HER3-transgenic mice to a BALB/c background (F1 Hybrid mice; BALB/c x MMTV-neu/MMTV-hHER3) and created a new human HER3 expressing tumor model based on the BALB/c-derived JC murine breast cancer cell line (JC-HER3).

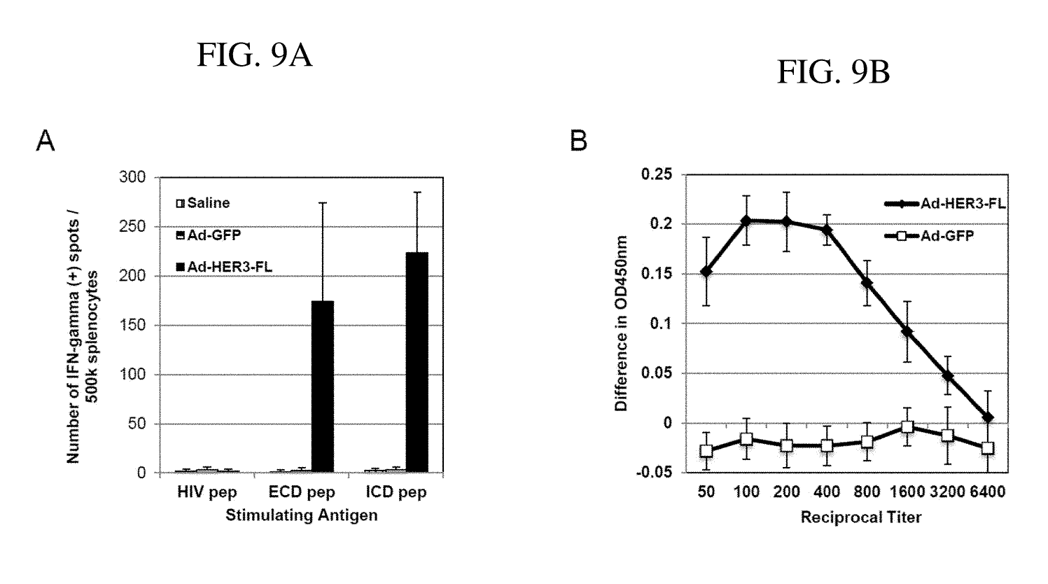

These human HER3-transgenic mice were immunized with the Ad-HER3-FL vector following which their splenocytes were analyzed for HER3-specific cellular immune responses by the IFN-gamma ELISPOT assay. FIG. 9A demonstrates an equally strong cellular response against epitopes from the HER3 extracellular domain (ECD) and intracellular domain (ICD) following Ad-HER3-FL vaccination. The vaccine also induced an anti-HER3 antibody response as measured by the binding of serum polyclonal antibodies to human HER3-transfected 4T1 murine breast cancer cells (4T1-HER3) compared with antibody binding after control Ad-GFP vaccination, FIG. 9B.

### Ad-HER3 Immunization Reduces Growth of Established HER3+ Breast Cancer

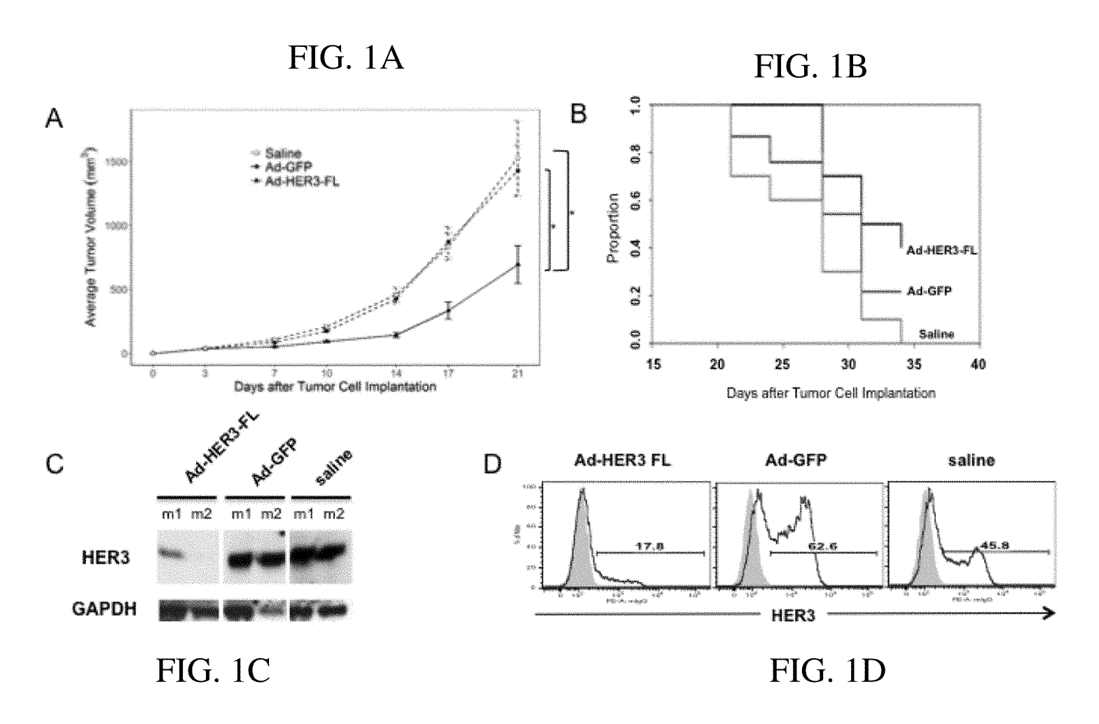

We tested the anti-tumor effects of vaccination with the Ad-HER3-FL construct in therapeutic models following JC-HER3 tumor cell implantation. We found that the Ad-HER3-FL vaccine effectively suppressed JC-HER3 tumor growth compared to the controls, specifically saline (p<0.001), and an irrelevant vaccine, Ad-GFP (p<0.001) (FIG. 1A), and this was associated with improved survival compared to saline treatment (p=0.005) (FIG. 1B) and demonstrated a trend toward improved survival when compared to the Ad-GFP vector, though we did not observe any tumor regression with Ad-HER3-FL vaccination.

In order to investigate potential sources for tumor escape from the HER3-specific immune response, we first analyzed tumor expression of HER3. In this model of HER3 immunotherapy, tumor expression of HER3 is not critical to maintaining the malignant phenotype. Therefore, one mechanism of immune escape in the presence of HER3 specific T cells and anti-HER3 antibodies would be HER3 antigen loss. We performed western blot on tumor lysates and flow cytometry on tumor cells remaining 21 days after the first vaccination. As shown in FIG. 1C, tumors from mice immunized with the Ad-HER3-FL vaccine, have down-regulation of HER3 expression, but it is not completely lost in all Ad-HER3-FL vaccinated mice. Similarly, on flow cytometric analysis, HER3 decreased but some HER3 expression persisted after Ad-HER3-FL vaccination (FIG. 1D). These data demonstrate that one mechanism of escape is antigen down regulation but it is not the only explanation.

### Ad-HER3-FL Vaccination Increases T Cell Infiltration into Tumors

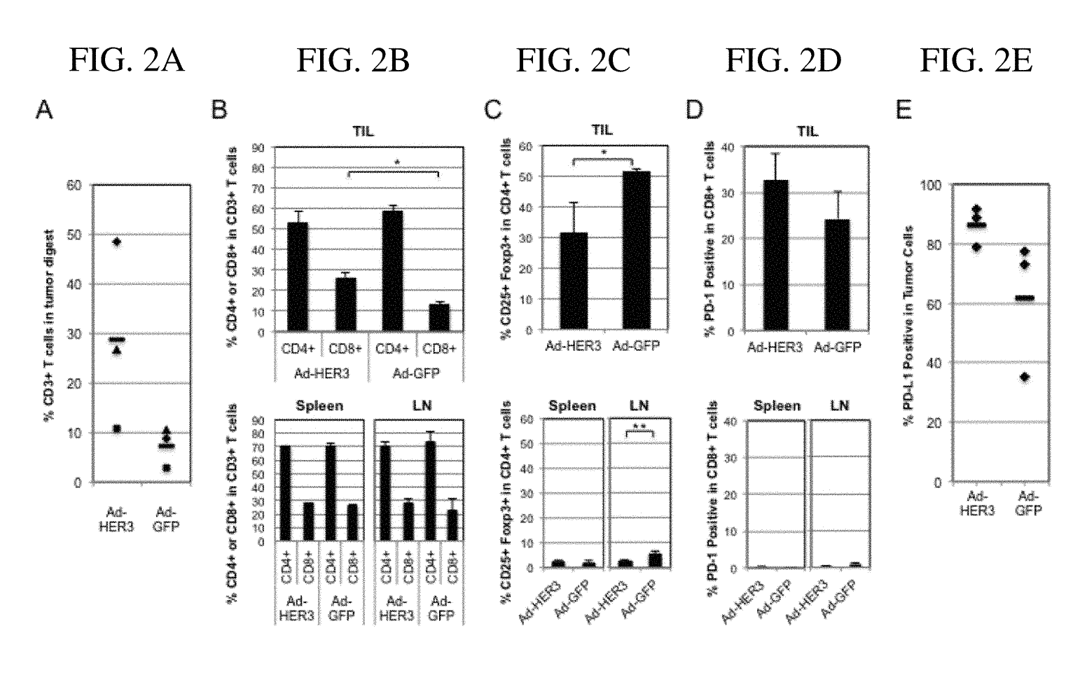

We sought to evaluate other potential explanations of tumor progression despite robust T cell responses against HER3. First we wished to determine if there was T cell infiltration of tumor by analyzing tumor infiltrating lymphocytes (TIL) in all vaccinated mice and found a greater number of CD3+ TILs in Ad-HER3-FL immunized mice compared to the Ad-GFP immunized mice (FIG. 2A). Among these TILs, there was a greater percentage of CD8+ (p<0.05) but not CD4+ TILs in the Ad-HER3-FL immunized mice. In contrast, there was no difference in the CD4+ and CD8+ T cell content within splenocytes or distant (non-tumor draining) lymph nodes in these Ad-HER3-FL vaccinated mice (FIG. 2B).

Other proposed mechanisms for immunosuppression involve the presence of regulatory T cells (Treg). We noted fewer intratumoral Tregs in the Ad-HER3-FL vaccinated mice compared to the Ad-GFP treated mice, p=0.026 (FIG. 2C), resulting in a greater intratumoral CD8+ to Treg ratio (data not shown). These data suggest that the immunosuppression did not involve activation of Tregs by the vaccine. Another well-established immunosuppressive mechanism is the presence of PD-1 on activated T cells.

Analysis of PD-1 expression on TILs, splenocytes and distant (non-tumor draining) lymph nodes after Ad-HERS-FL or Ad-GFP vaccination confirmed that PD-1 tended to be overexpressed by CD8+ TILs after Ad-HER3-FL vaccination compared to PD-1 expression by CD8+ T cells isolated from splenocytes and non-tumor draining lymph nodes in these same mice (FIG. 2D). Similarly, we noted a trend for higher tumor cell PD-L1 expression after Ad-HER3-FL vaccination compared to control, FIG. 2E. These data suggest that activated TILs induced by Ad-HER3-FL vaccination are at risk of being suppressed through the PD-1/PD-L1 signaling axis due to both tumor PD-L1 expression and their own high PD-1 expression.

### Enhanced Antitumor Activity with Checkpoint Blockade Plus Ad-HER3 Vaccine

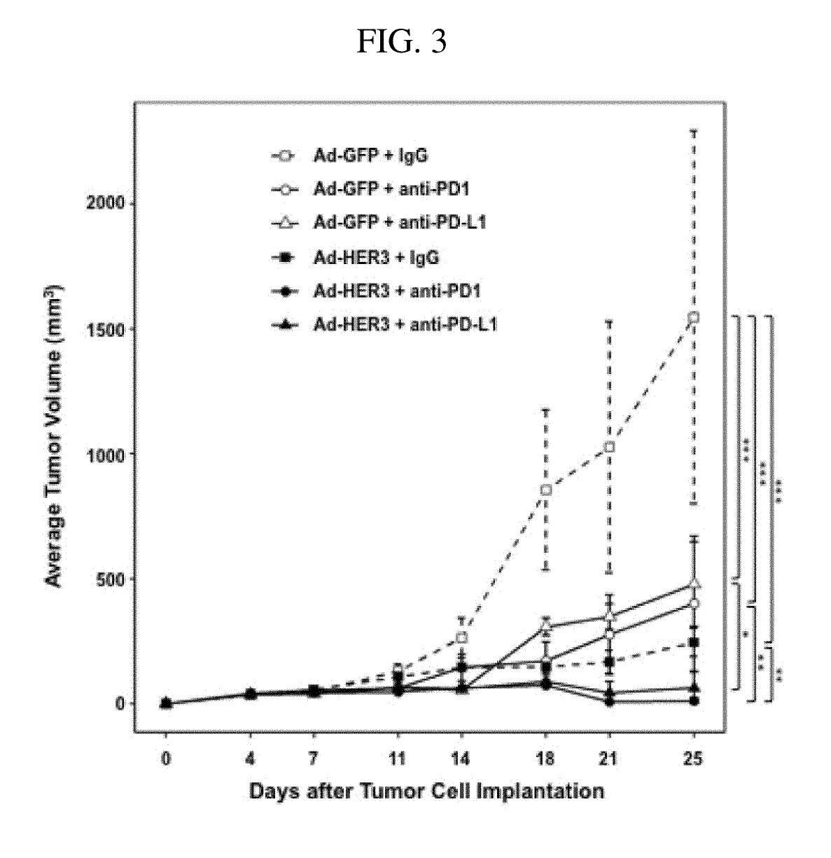

In order to study the functional consequences of PD-1 expression by intratumoral T cells, we tested whether blockade of the PD-1/PD-L1 interaction in combination with Ad-HER3-FL immunizations would have greater anti-tumor efficacy than either alone. We first evaluated this effect in a tumor prevention model. In this model, mice were first immunized with the Ad-HER3-FL vaccine, tumor was then implanted, and tumor implantation was followed by anti-PD-1 or anti-PD-L1 antibody administration. While Ad-HER3-FL alone or anti-PD-1 or anti-PD-L1 with control vector resulted in some delayed tumor growth, there was no tumor regression (FIG. 3). In contrast, vaccination with Ad-HER3-FL prior to tumor implantation followed by either anti-PD-L1 or anti-PD-1 antibodies after tumor implantation induced tumor regression (p<0.01 for the comparison of Ad-HER3-FL+IgG versus Ad-HER-FL+anti-PD1; p<0.01 for the comparison of Ad-HER3-FL+IgG versus Ad-HER3-FL+anti-PD-L1).

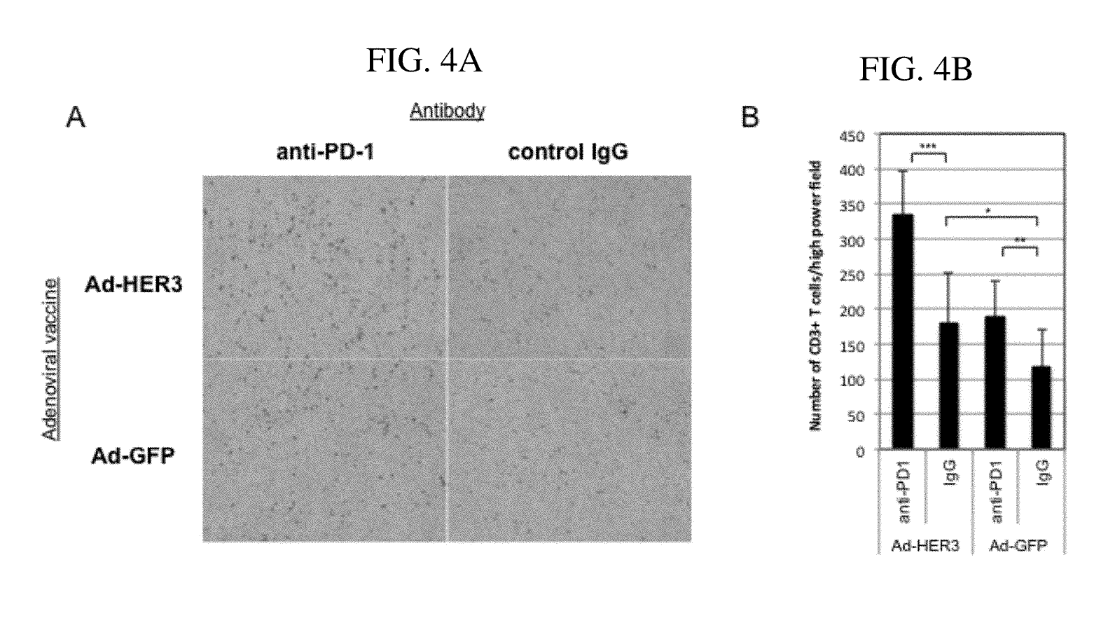

We next wanted to determine whether tumor regression was due to the modulation of the intratumoral T cell infiltrate by checkpoint blockade after vaccination in the prevention model. The addition of anti-PD-1 antibodies to Ad-HER3-FL vaccination significantly increased the number of CD3+ T cells/hpf within the tumor compared to Ad-HER3-FL vaccination alone (p<0.0001) (FIGS. 4A, 4B). Interestingly, there was an increase in the T cell infiltrate caused by anti-PD-1 antibody treatment regardless of whether the anti-PD-1 antibody was administered with either Ad-HER3-FL or Ad-GFP. However, the combination of Ad-HER3-FL plus anti-PD-1 antibody induced the greatest T cell infiltrate/hpf.

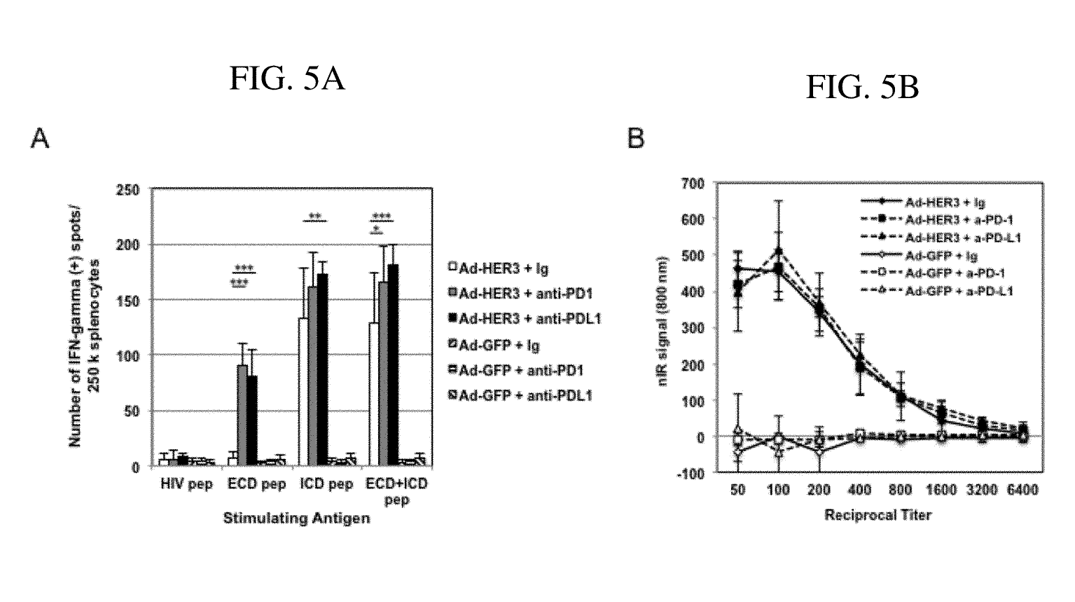

We next interrogated if anti-PD-1 treatment could augment the magnitude of both the HER3-specific T cell and anti-HER3 antibody response induced by Ad-HER3-FL alone. In the prevention model, splenocytes from mice treated with either the Ad-HER3-FL vaccine, Ad-HER3-FL vaccine+anti-PD-1 antibody, or Ad-HER3-FL vaccine+anti-PD-L1 antibody demonstrated an increased frequency of T cells specific for HER3 ECD and ICD peptides (FIG. 5A). However, neither anti-PD-1 nor anti-PD-L1 antibodies given with control vaccine affected the serum titer of anti-HER3 antibodies induced by Ad-HER3-FL-immunization (FIG. 5B). These data support a role for PD-1/PD-L1 blockade as an additional strategy to further increase antigen-specific T cell activation induced by Ad-HER3-FL vaccination.

### Combination of Checkpoint Blockade and Ad-HER3-FL has Enhanced Anti-Tumor Activity in Tumor Bearing Mice

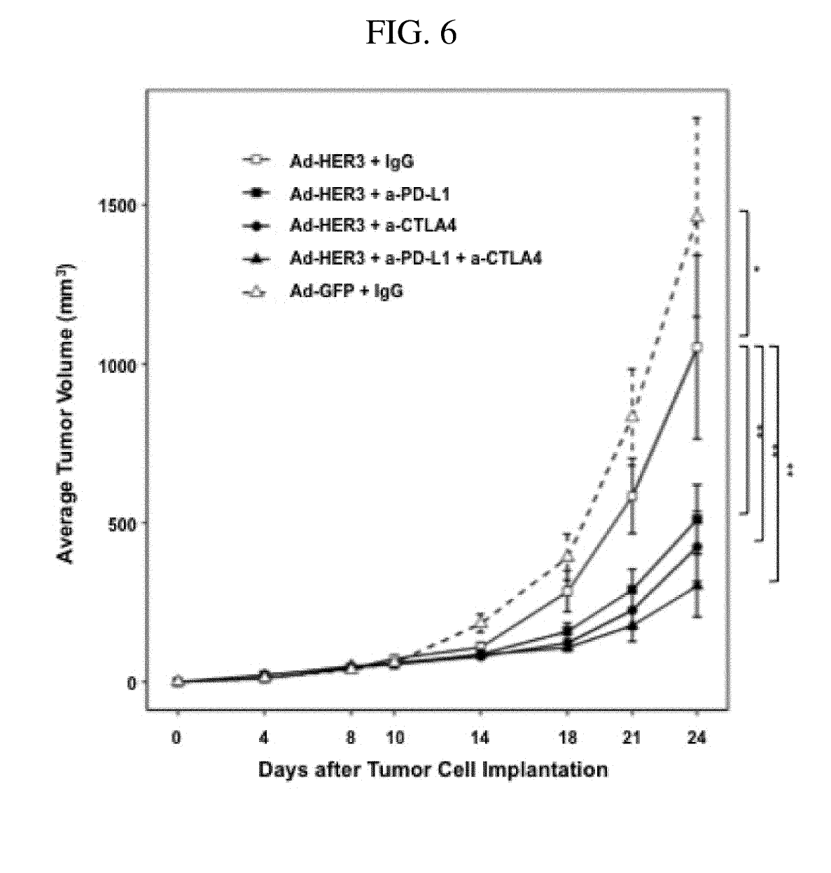

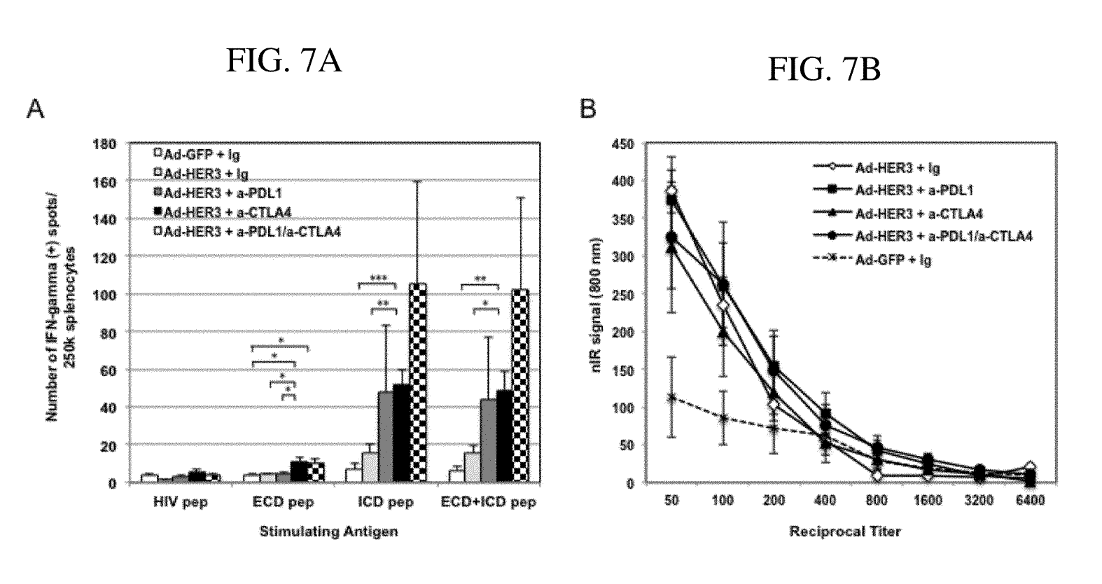

Having demonstrated that checkpoint blockade enhanced the anti-tumor activity of the Ad-HER3-FL in the less stringent prevention model, we wished to evaluate the efficacy of these antibodies in enhancing the anti-tumor activity of Ad-HER3-FL immunization in tumor-bearing mice (treatment model). We focused on anti-PD-L1 and anti-CTLA4 in these experiments. HER3 transgenic mice implanted with JC-HER3 cells were vaccinated with Ad-HER3-FL or control Ad-GFP simultaneously with anti-PD-L1, anti-CTLA4 or both. There was slowing of tumor growth by Ad-HER3-FL plus either antibody alone (p<0.001, for both comparisons) or with the combination of both antibodies (p<0.001) compared with Ad-HER3-FL alone (FIG. 6). Analysis of splenocytes from this experiment suggested that anti-PD-L1 or anti-CTLA4 or their combination plus the HER3 vaccine increased the magnitude of HER3-specific T cell response compared with vaccine alone (FIG. 7A). Furthermore, there was no apparent difference in the titer of antibodies induced with Ad-HER3 vaccine with or without the addition of the checkpoint antibodies (FIG. 7B).

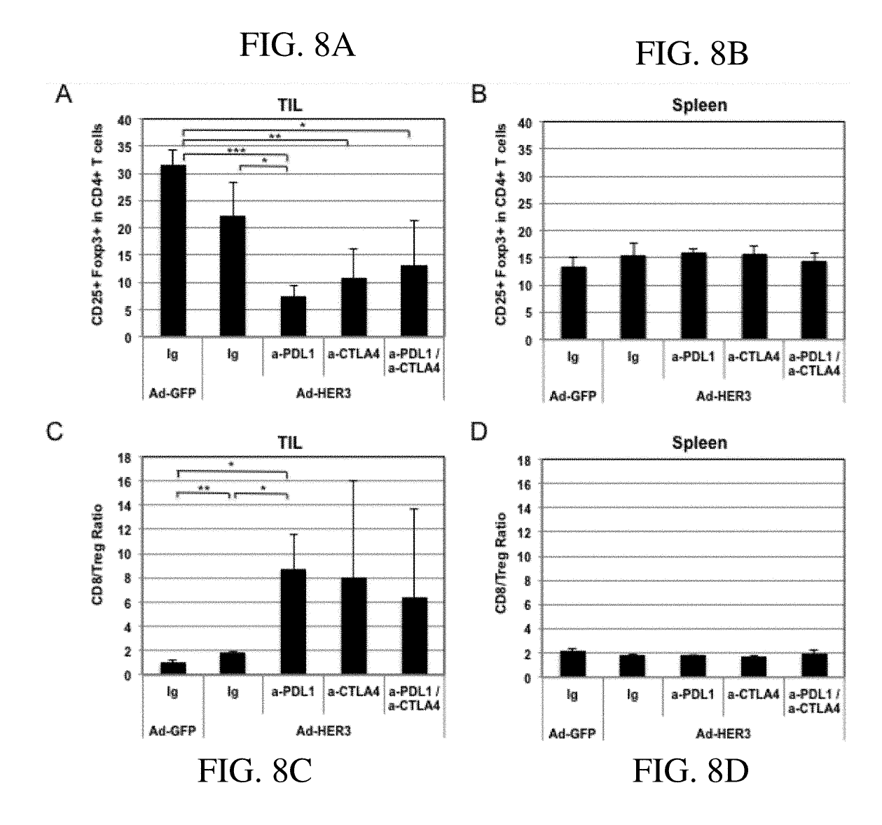

Further, each antibody and their combination when administered with the Ad-HER3-FL vaccine, decreased intratumoral Treg content (FIG. 8A) and increased CD8 to Treg ratio (FIG. 8C) in established tumors compared with Ad-HER3-FL alone. In contrast, there was no significant difference in the splenic Treg content (FIG. 8B) or CD8 to Treg ratio (FIG. 8D) when comparing the different treatment conditions, suggesting that the effect of the checkpoint antibodies occurs at the site of the tumor.

### Discussion

HER3 mediates resistance to EGFR-, HER2- and endocrine-directed therapies in breast cancer and other epithelial malignancies, but has been challenging to target. Our initial objective was to develop a vaccine capable of inducing HER3-specific immune effectors, which would have anti-tumor efficacy against resistant tumors. We chose an adenoviral backbone deleted of the E1 and E2b genes that we previously demonstrated in clinical studies to activate immune responses against the encoded transgene despite the development of anti-Ad neutralizing antibody (30). We developed a model of human HER3 expressing murine breast cancer (JC-HER3) implantable into immune competent human HER3 transgenic mice to test the adenoviral vaccines. The E1, E2b-deleted vector induced T cells with specificities against both intracellular and extracellular domains of HER3 in HER3-transgenic mice. The Ad-HER3 vaccine also demonstrated the ability to modulate the immune cell content of tumors. Specifically, Ad-HER3 vaccination resulted in an increased percentage of intratumoral CD8 T cells and a decreased percentage of intratumoral Tregs, yielding an increased CD8 to Treg ratio, a trend favorable for inducing immune mediated anti-tumor activity. This resulted in a delay in tumor growth; however, we wished to develop a strategy that led to greater tumor regression.

One strategy to enhance the antitumor activity of the vaccine was suggested by the observation that although the Ad-HER3-FL immunization caused an increase in TILs compared to control immunizations, these TILs demonstrated high expression of PD-1 compared with splenocytes or T cells from non-tumor draining lymph nodes. It has been previously suggested that T cells specific for a vaccinating antigen upregulate PD-1.(30, 31) As the PD-1/PD-L1 interaction is well established to impair T cell-mediated anti-tumor activity, we sought to enhance the anti-tumor activity of the Ad-HER3-FL vaccine by blocking the PD-1/PD-L1 interaction. Indeed, there was elimination of tumor when we immunized mice with the Ad-HER3-FL prior to tumor implantation and then delivered the anti-PD-1 or anti-PD-L1 antibody after tumor implantation. In this setting, there was sufficient time to generate a robust intratumoral antigen specific immune response which could be further enhanced by checkpoint blockade. The robust immune response generated by vaccination before tumor cell implantation may model the clinical scenario of vaccination of patients with resected tumors at high risk of recurrence. In this setting if tumor were to recur, anti-PD-1/PD-L1 blockade may lead to tumor regression because of the presence of intratumoral T cells activated by previous vaccination. This may also model the clinical scenario of tumors controlled by standard therapy, which then grow upon development of resistance due to upregulation of molecules such as HER3. In this setting, tumors that upregulate HER3 and contain infiltrates with HER3 specific T cells would be rapidly eliminated upon application of PD-1/PD-L1 blockade.

In contrast to the prevention model, vaccination therapies of established malignancies have had modest success in pre-clinical and clinical testing; as other groups have reported greater anti-tumor activity for vaccines combined with PD-1/PD-L1 blockade in murine treatment models (32-34), we wished to test the administration of PD-1/PD-L1 blockade with Ad-HER3-FL in established tumors. In the stringent treatment models, there was slowing of tumor growth with either PD-1/PD-L1 blockade. We reasoned that in treatment models, there would be little time for a T cell response following vaccination alone to achieve a frequency necessary to eradicate tumor. Therefore, we also tested the addition of anti-CTLA4 to determine if this alone or in conjunction with PD-1/PD-L1 blockade could cause rapid T cell expansion after vaccination.

In poorly immunogenic tumor models, it has been demonstrated that anti-CTLA4 therapy strongly enhances the amplitude of vaccine induced anti-tumor activity (35, 36). We observed in the treatment model that anti-CTLA4 or blockade of the PD-1/PD-L1 interaction (anti-PD-L1) and their combination plus the Ad-HEr3 vaccine similarly enhanced immune-mediated tumor control.

Our data suggest that current cancer vaccine strategies would be enhanced by checkpoint blockade. Single and dual checkpoint blockade appear to enhance anti-tumor response to the Ad-HER3 vaccine similarly. Therefore, the choice of checkpoint antibody may depend more on their indication. For example, if single agent checkpoint blockade is the standard therapy for a malignancy where HER3 would also be relevant (e.g. triple negative breast cancer), then combining the HER3 vaccine with the standard single agent checkpoint blockade antibody would be appropriate. However, where dual checkpoint is the standard, our data suggest that this leads to similar enhancement in anti-tumor activity to the HER3-FL vaccine.

Our data now warrant clinical testing of the Ad-HER3-FL vaccine with anti-PD-1/PD-L1, anti-CTLA4 therapy, or both in the setting of established malignancy and with anti-PD-1 or anti-PD-L1 antibodies in the adjuvant setting. As our pre-clinical testing has demonstrated minimal side effects from this vaccine, we anticipate that our planned first-in-human clinical trial of this vaccine will be well tolerated. A phase I study of the Ad-HER3 full-length vaccine will open shortly in order to evaluate the safety and immunogenicity of this vaccine in metastatic cancer patients with a planned expansion cohort for hormone receptor positive breast cancer. As HER3 is recognized to mediate anti-HER2 therapy resistance, we plan to open a clinical trial of the Ad-HER3 vaccine given in combination with anti-HER2 therapy in metastatic HER2+ breast cancer. Our prior studies have also revealed that in HER2+ breast cancer, activation of the HER3 signaling axis is associated with a poor outcome (37). Lastly, there is increasing evidence that single agent check point blockade is clinically active in a portion of TNBC patients (38, 39). In addition, there is evidence that HER3 expression is associated with worse DFS and OS in TNBC (40). Based on these observations, we will open a trial of concurrent Ad-HER3 vaccination and check point blockade in TNBC to assess the safety and immunogenicity of this combination therapy.

### Materials and Methods

### Adenoviral Vector Preparation

The human HER3 cDNA was excised from a pCMVSport6-HER3-HsIMAGE6147464 plasmid (cDNA clone MGC:88033/IMAGE:6147464) from the ATCC (Manassas, Va.). Construction of a first-generation [E1−, E3−] Ad vector containing human full length HER3 under control of human CMV promoter/enhancer elements was performed using the pAdEasy system (Agilent technologies, Santa Clara, Calif.) as previously described(41). The modified adenoviral vector, [E1−,E2b−] Ad, was constructed as previously described (42). This vector has multiple deletions of the early region 1 (E1) and E2b regions (DNA polymerase and pTP genes), and was engineered to express the identical human CMV promoter/enhancer-transgene cassette as utilized for the [E1⁻E3⁻] Ad-HER3 vector. Ad[E1−E2b−]-HER3 FL vector was constructed with full length of HER3 cDNA. Complementing C-7 cell lines were used to support the growth and production of high titers of these vectors, and cesium chloride double banding was performed to purify the vectors, as previously reported (43).

### Reagents and Peptides

Mixtures of HER3 peptides containing 15 mer peptides, each overlapping the next by 11 amino acids, spanning extracellular domain plus transmembrane segment (ECD-TM) of HER3 protein and intracellular domain (ICD) of HER3 protein, were purchased from JPT Peptide Technologies (Berlin, Germany), and were used for the IFN-γ ELISPOT assay. An HIV peptide mix representing HIV gag protein was purchased from JPT Peptide Technologies (Berlin, Germany) and was used as a negative control. Anti-murine PD-1 (BE0146, clone J43) and anti-murine PD-L1 (BE0101, clone 10F.9G2) and anti-murine CTLA4 (BE0164, clone 9D9) monoclonal antibodies were purchased from Bio X Cell (West Lebanon, N.H.) for animal experiments. Collagenase III (cat# 4183) was purchased from Worthington Biochemical (Lakewood, N.J.), and hyaluronidase (H3884) and DNase (D5025) from Sigma-Aldrich (St. Louis, Mo.).

### Mice

Female wild-type BALB/c mice (Jackson Laboratory, Bar Harbor, Me., USA) were bred and maintained in the Duke University Medical Center pathogen-free Animal Research Facility, and used at 6 to 8 weeks of age. Human HER3-transgenic mice (MMTV-neu/MMTV-hHER3) with FVB background were a kind gift from Dr. Stan Gerson at Case Western Reserve University. FVB mice homozygous for the HER3 gene were established at Duke University and crossed with BALB/c mice to generate F1 hybrid HER3 transgenic mice (FVB x BALB/c) for use in tumor implantation experiments. All animal studies described were approved by the Duke University Medical Center Institutional Animal Care & Use Committee and the US Army Medical Research and Materiel Command (USAMRMC) Animal Care and Use Review Office (ACURO) and performed in accordance with guidelines published by the Commission on Life Sciences of the National Research Council.

### Detecting HER3 Expression by Western Blotting

Tumor tissues were collected at the termination of animal experiments and minced and homogenized in RIPA buffer in the presence of proteinase inhibitors. After centrifugation at 13,000 rpm for 10 min at 4° C., the supernatant was pooled, filtered through a 0.22 μm filter, aliquoted and stored at −80° C. until needed. Protein concentration was determined by a BCA assay. Thirty μg of protein was applied for each lane, run on 12% Tris-HCl acrylamide gel, and transferred to polyvinylidene fluoride (PVDF) membranes. Membranes were incubated with anti-HER3 antibody (1:1000 dilution, Santa Cruz Biotechnology, Inc., Santa Cruz, Calif.) or anti-GAPDH antibody (1:1000 dilution, Santa Cruz) for 1 h, followed by incubation with horseradish peroxidase-conjugated goat anti-mouse IgG antibody (1:2000 dilution, Bio-Rad, Hercules, Calif.). The chemiluminescent substrate kit (Thermo Scientific, Rockford, Ill.) was used for the development.

### Flow Cytometry of Tumor Infiltrating Lymphocytes and Tumor HER3 Expression

Tumors were excised from mice at the termination of tumor implantation experiments, minced with surgical blades and digested with triple enzyme buffer (collagenase III, hyaluronidase, DNase) for 1.5 hours at 37° C. The cell suspension was washed 3 times with PBS and resuspended in PBS. Cells were first labeled with viability dye (Fixable Aqua Dead Cell Stain Kit, Invitrogen, Eugene, Oreg.) for 5 min, and then with PerCP/Cy5.5-anti-CD3, APC/Cy7-anti-CD8, Alexa Fluor 700-anti-CD4, FITC-anti-CD25, APC-anti-PD-1, and PE-anti-PD-L1 or PE-anti-HER3 antibody (BioLegend, San Diego, CA) for 30 min at 4° C. Cells were washed twice with PBS and analyzed on a LSRII machine (BD Biosciences) using FlowJo software.

### IFN-γ Enzyme-Linked Immunosorbent Spot (ELISpot) Assay

Mouse IFN-γ ELISPOT assay (Mabtech Inc., Cincinnati, Ohio) was performed according to the manufacturer's instructions. At the end of the mouse experiments, their spleens were collected and lymphocytes were harvested by mincing and passing through a 40 μm Cell Strainer. Red blood cells were lysed with red blood cell lysis buffer (Sigma). Splenocytes (500,000 cells/well) were incubated in RPMI-1640 medium (Invitrogen) supplemented with 10% horse serum, and HER3 ECD-TM peptide mix and/or HER3 ICD peptide mix (1.3 μg/ml) were used as stimulating antigens. HIV peptide mix was used as a negative control, and a mixture of PMA (50 ng/ml) and Ionomycin (1 μg/ml) was used as a positive control for the assay. Membranes were read with a high-resolution automated ELISpot reader system (Carl Zeiss, Inc., Thornwood, N.Y., USA) using the KS ELISpot version 4.2 software.

### Cell-Based ELISA

4T1 cells were transduced with HER3 gene by lentiviral vectors to express human HER3 on the cell surface (4T1-HER3 cell). 4T1 and 4T1-HER3 cells were incubated overnight at 37° C. in 96 well flat bottomed plates (3×10⁴ cells in 100 μL medium/well). Mouse sera were prepared by diluting with DMEM medium (final titrations 1:50˜1:6,400), and 50 μl of mouse sera-containing media were added to the wells and incubated for 1 hour on ice. The plates were gently washed with PBS twice, and then, cells were fixed with diluted formalin (1:10 dilution of formalin in 1% BSA in PBS) for 20 min at room temperature. After washing three times with PBS, 50 μL of 1:2000 diluted HRP-conjugated goat anti-mouse IgG was added to the wells, and incubated for 1 h at room temperature. After washing three times with PBS, TMB substrate was added to the wells (50 μl/well) and incubated for approximately 20 min. The color development was stopped by adding 50 μl of 1M H₂SO₄ buffer. Absorbance at 450 nm was read using a BioRad Microplate Reader (Model 680). As the alternative method for the detection of HER3-specific antibody, near infrared red (nIR) dye-conjugated anti-mouse IgG (IRDye 800CW, LI-COR Biosciences, Lincoln, Nebr.) was used as a secondary antibody, and the nIR signal was detected by a LI-COR Odyssey Imager (LI-COR) using the 800 nm channel.

### Prophylactic Anti-Tumor Model in HER3-Transgenic Mice

HER3-transgenic F1 hybrid mice were immunized by footpad injection on days −11, −4 and 14 with 2.6×10¹⁰ particles of the Ad[E1−,E2b−]-HER3-FL or Ad-GFP control in 40 μL of saline. On day 0, mice were inoculated with 5×10⁵ JC-HER3 cells in 100 μl saline subcutaneously into the flank. Tumor dimensions were measured serially, and tumor volumes calculated using the following formula: long axis×(short axis)²×0.5. For the combination treatment with immune checkpoint inhibitors, mice were vaccinated with Ad-HER3-FL or Ad-GFP on days −11, −4 and 14, and received peritoneal injection of anti-PD-1 antibody, anti-PD-L1 antibody or control IgG (200 μg/injection) twice a week (on days 3, 6, 10, 13, 17 and 20) after tumor implantation.

### Therapeutic Anti-Tumor Model in F1 Hybrid HER3 Transgenic Mice

HER3 transgenic F1 hybrid mice were inoculated with 5×10⁵ JC-HER3 cells in 100 μL saline subcutaneously into the flank on day 0. On days 3 and 10, mice were immunized via footpad injection with Ad-HER3-FL or Ad-GFP control vector (2.6×10¹⁰ particles/mouse for each injection). Tumor dimensions were measured serially and tumor volumes were calculated as described above. Mice were euthanized when the tumor size reached the humane endpoint, or by day 34. For the combined treatment with immune checkpoint inhibitors (anti-PD-1, anti-PD-L1, or anti-CTLA4 antibody), mice received peritoneal injection of the checkpoint inhibitor (200 μg/injection) twice a week after tumor implantation.

### Tissue Analysis of Tumor-Infiltrating T Cells

Tumor tissue collected at the time mice were euthanized was fixed in 10% neutral buffered formalin for a minimum of 24 hours. The tissue was then processed and embedded in paraffin. Sections with 5 μm thickness were made for hemotoxylin and eosin staining and CD3+ T cell staining. For immunohistochemistry using anti-CD3 antibody (Thermo Fisher Scientific, Waltham, Mass.), heat-induced antigen retrieval was performed using sodium citrate buffer for 20 min after deparaffinization of tissue sections. Following quenching of endogenous peroxidase activity with 3% H₂O₂, 10% normal horse serum was used to block nonspecific binding sites. Anti-CD3 antibody (1:150 dilution) was applied to the sections, which were incubated overnight at 4° C. After three washes with PBS, anti-rabbit IgG secondary antibody (ImmPRESS anti-Rabbit IgG Polymer, Vector Lab, Burlingame, Calif.) was applied for 30 min, and then color was developed using the DAB Peroxidase substrate kit (Vector Lab). Counterstaining was performed with hematoxylin. After assessment of adequate staining by two independent observers, ten high power fields (magnification ×200; objective lens ×20, ocular ×10) of tumor tissue for each group, avoiding necrotic area, were randomly selected and photographed using an IX73 Inverted Microscope with Dual CCD Chip Monochrome/Color Camera (Olympus). CD3-positive spots were counted for each field by two observers who had no previous knowledge of treatments performed for individual groups.

### Statistical analysis

For the ELISpot and ELISA assays, differences in IFN-γ production and antibody binding, respectively, were analyzed using the Student's t test. Tumor volume measurements for in vivo models were analyzed under a cubic root transformation to stabilize the variance. Welch t-tests were used to assess differences between mice injected with HER3-VIA or control GFP-VIA.

To compare tumor growth volumes over time, a multivariable Generalized Additive Model for Location, Scale and Shape (GAMLSS) (44) considering Group, Experiment, Time and interaction between Time and Group as covariables for Tumor Volume location and Time for Tumor Volume scale was applied. The Normal distribution was considered for the effectiveness of Ad-HER3 FL vaccine model and the Zero Adjusted Gamma distribution for the effectiveness of antibodies model. Time was modeled using penalized cubic spline (45) and the interaction between Time and Group was modeled using Varying Coefficient (46). Areas under tumor growth curve were calculated under spline interpolation (44) and adaptive quadrature for the tumor prevention model. A simple GAMLSS with Gamma distribution quantifies the relationship between mean of area under tumor growth curve and the covariable Group.

Contrasts were calculated using the Wald statistic and multiples comparisons were corrected as suggested by Holm (47). Model diagnostics was performed based on Worm-plots (48) and fitted values were compared considering 95% Bootstrap Confidence Intervals (49).

The Kaplan-Meier method was used to estimate overall survival and treatments were compared using a two-sided log-rank test. Analyses were performed using R version 2.10.1, SAS v. 9.3 (SAS Institute, Cary NC) and R, version 3.2.5 (50), survival plots were created using Spotfire S+ v. 8.1 (TIBCO, Palo Alto, Calif.). All tests of hypotheses will be two-sided considering a significance level of 0.05.

### REFERENCES

1. Roskoski R, Jr. The ErbB/HER family of protein-tyrosine kinases and cancer. Pharmacological research: the official journal of the Italian Pharmacological Society. 2014; 79:34-74.
2. Cai Z, Zhang H, Liu J, Berezov A, Murali R, Wang Q, et al. Targeting erbB receptors. Seminars in cell & developmental biology. 2010; 21(9):961-6.
3. Takikita M, Xie R, Chung J Y, Cho H, Ylaya K, Hong S M, et al. Membranous expression of Her3 is associated with a decreased survival in head and neck squamous cell carcinoma. J Transl Med. 2011; 9:126.
4. Chiu C G, Masoudi H, Leung S, Voduc D K, Gilks B, Huntsman D G, et al. HER-3 overexpression is prognostic of reduced breast cancer survival: a study of 4046 patients. Annals of surgery. 2010; 251(6):1107-16.
5. Hayashi M, Inokuchi M, Takagi Y, Yamada H, Kojima K, Kumagai J, et al. High expression of HER3 is associated with a decreased survival in gastric cancer. Clin Cancer Res. 2008; 14(23):7843-9.
6. Giltnane J M, Moeder C B, Camp R L, Rimm D L. Quantitative multiplexed analysis of ErbB family coexpression for primary breast cancer prognosis in a large retrospective cohort. Cancer. 2009; 115(11):2400-9.
7. Begnami M D, Fukuda E, Fregnani J H, Nonogaki S, Montagnini A L, da Costa W L, Jr., et al. Prognostic implications of altered human epidermal growth factor receptors (HERs) in gastric carcinomas: HER2 and HER3 are predictors of poor outcome. J Clin Oncol. 2011; 29(22):3030-6.
8. Reschke M, Mihic-Probst D, van der Horst E H, Knyazev P, Wild P J, Hutterer M, et al. HER3 is a determinant for poor prognosis in melanoma. Clin Cancer Res. 2008; 14(16):5188-97.
9. Lee C H, Huntsman D G, Cheang M C, Parker R L, Brown L, Hoskins P, et al. Assessment of Her-1, Her-2, And Her-3 expression and Her-2 amplification in advanced stage ovarian carcinoma. Int J Gynecol Pathol. 2005; 24(2):147-52.
10. Kol A, Terwisscha van Scheltinga A G, Timmer-Bosscha H, Lamberts L E, Bensch F, de Vries E G, et al. HER3, serious partner in crime: therapeutic approaches and potential biomarkers for effect of HER3-targeting. Pharmacol Ther. 2014; 143(1):1-11.
11. Sergina N V, Rausch M, Wang D, Blair J, Hann B, Shokat K M, et al. Escape from HER-family tyrosine kinase inhibitor therapy by the kinase-inactive HER3. Nature. 2007; 445(7126):437-41.
12. Frogne T, Benjaminsen R V, Sonne-Hansen K, Sorensen B S, Nexo E, Laenkholm A V, et al. Activation of ErbB3, EGFR and Erk is essential for growth of human breast cancer cell lines with acquired resistance to fulvestrant. Breast Cancer Res Treat. 2009; 114(2):263-75.
13. Musgrove E A, Sutherland R L. Biological determinants of endocrine resistance in breast cancer. Nat Rev Cancer. 2009; 9(9):631-43.
14. Tovey S, Dunne B, Witton C J, Forsyth A, Cooke T G, Bartlett J M. Can molecular markers predict when to implement treatment with aromatase inhibitors in invasive breast cancer? Clin Cancer Res. 2005; 11(13):4835-42.
15. Arpino G, Wiechmann L, Osborne C K, Schiff R. Crosstalk between the estrogen receptor and the HER tyrosine kinase receptor family: molecular mechanism and clinical implications for endocrine therapy resistance. Endocr Rev. 2008; 29(2):217-33.
16. Liu B, Ordonez-Ercan D, Fan Z, Edgerton S M, Yang X, Thor A D. Downregulation of erbB3 abrogates erbB2-mediated tamoxifen resistance in breast cancer cells. Int J Cancer. 2007; 120(9):1874-82.
17. Hsieh A C, Moasser M M. Targeting HER proteins in cancer therapy and the role of the non-target HER3. Br J Cancer. 2007; 97(4):453-7.
18. Folgiero V, Avetrani P, Bon G, Di Carlo S E, Fabi A, Nistico C, et al. Induction of ErbB-3 expression by alpha6beta4 integrin contributes to tamoxifen resistance in ERbetal-negative breast carcinomas. PLoS One. 2008; 3(2):e1592.
19. Frogne T, Jepsen J S, Larsen S S, Fog C K, Brockdorff B L, Lykkesfeldt A E. Antiestrogen-resistant human breast cancer cells require activated protein kinase B/Akt for growth. Endocr Relat Cancer. 2005; 12(3):599-614.
20. Gala K, Chandarlapaty S. Molecular pathways: HER3 targeted therapy. Clin Cancer Res. 2014; 20(6):1410-6.
21. Sakai K, Yokote H, Murakami-Murofushi K, Tamura T, Saijo N, Nishio K. Pertuzumab, a novel HER dimerization inhibitor, inhibits the growth of human lung cancer cells mediated by the HER3 signaling pathway. Cancer Sci. 2007; 98(9):1498-503.
22. Cai Z, Zhang G, Zhou Z, Bembas K, Drebin J A, Greene M I, et al. Differential binding patterns of monoclonal antibody 2C4 to the ErbB3-p185her2/neu and the EGFR-p185her2/neu complexes. Oncogene. 2008; 27(27):3870-4.
23. Junttila T T, Akita R W, Parsons K, Fields C, Lewis Phillips G D, Friedman L S, et al. Ligand-independent HER2/HER3/PI3K complex is disrupted by trastuzumab and is effectively inhibited by the PI3K inhibitor GDC-0941. Cancer Cell. 2009; 15(5):429-40.
24. Ren X R, Wei J, Lei G, Wang J, Lu J, Xia W, et al. Polyclonal HER2-specific antibodies induced by vaccination mediate receptor internalization and degradation in tumor cells. Breast cancer research: BCR. 2012; 14(3):R89.
25. Stamper C C, Zhang Y, Tobin J F, Erbe D V, Ikemizu S, Davis S J, et al. Crystal structure of the B7-1/CTLA-4 complex that inhibits human immune responses. Nature. 2001; 410(6828):608-11.
26. Topalian S L, Drake C G, Pardoll D M. Immune checkpoint blockade: a common denominator approach to cancer therapy. Cancer Cell. 2015; 27(4):450-61.
27. Shin D S, Ribas A. The evolution of checkpoint blockade as a cancer therapy: what's here, what's next? Curr Opin Immunol. 2015; 33:23-35.
28. Tumeh P C, Harview C L, Yearley J H, Shintaku I P, Taylor E J, Robert L, et al. PD-1 blockade induces responses by inhibiting adaptive immune resistance. Nature. 2014; 515(7528):568-71.
29. Osada T, Yang X Y, Hartman Z C, Glass O, Hodges B L, Niedzwiecki D, et al. Optimization of vaccine responses with an E1, E2b and E3-deleted Ad5 vector circumvents pre-existing anti-vector immunity. Cancer Gene Ther. 2009; 16(9):673-82.
30. Fourcade J, Sun Z, Pagliano O, Chauvin J M, Sander C, Janjic B, et al. PD-1 and Tim-3 regulate the expansion of tumor antigen-specific CD8(+) T cells induced by melanoma vaccines. Cancer Res. 2014; 74(4):1045-55.
31. Wong R M, Scotland R R, Lau R L, Wang C, Korman A J, Kast W M, et al. Programmed death-1 blockade enhances expansion and functional capacity of human melanoma antigen-specific CTLs. International immunology. 2007; 19(10):1223-34.
32. Karyampudi L, Lamichhane P, Scheid A D, Kalli K R, Shreeder B, Krempski J W, et al. Accumulation of memory precursor CD8 T cells in regressing tumors following combination therapy with vaccine and anti-PD-1 antibody. Cancer Res. 2014; 74(11):2974-85.
33. Soares K C, Rucki A A, Wu A A, Olino K, Xiao Q, Chai Y, et al. PD-1/PD-L1 blockade together with vaccine therapy facilitates effector T-cell infiltration into pancreatic tumors. J Immunother. 2015; 38(1):1-11.
34. Binder D C, Engels B, Arina A, Yu P, Slauch J M, Fu Y X, et al. Antigen-specific bacterial vaccine combined with anti-PD-L1 rescues dysfunctional endogenous T cells to reject long-established cancer. Cancer immunology research. 2013; 1(2):123-33.
35. van Elsas A, Hurwitz A A, Allison J P. Combination immunotherapy of B16 melanoma using anti-cytotoxic T lymphocyte-associated antigen 4 (CTLA-4) and granulocyte/macrophage colony-stimulating factor (GM-CSF)-producing vaccines induces rejection of subcutaneous and metastatic tumors accompanied by autoimmune depigmentation. J Exp Med. 1999; 190(3):355-66.
36. Li B, VanRoey M, Wang C, Chen T H, Korman A, Jooss K. Anti-programmed death-1 synergizes with granulocyte macrophage colony-stimulating factor-secreting tumor cell immunotherapy providing therapeutic benefit to mice with established tumors. Clin Cancer Res. 2009; 15(5):1623-34.
37. Xia W, Petricoin E F, 3rd, Zhao S, Liu L, Osada T, Cheng Q, et al. An heregulin-EGFR-HER3 autocrine signaling axis can mediate acquired lapatinib resistance in HER2+breast cancer models. Breast cancer research: BCR. 2013; 15(5):R85.
38. Nanda R, Chow L Q, Dees E C, Berger R, Gupta S, Geva R, et al. Pembrolizumab in Patients With Advanced Triple-Negative Breast Cancer: Phase Ib KEYNOTE-012 Study. J Clin Oncol. 2016; 34(21):2460-7.
39. Emens L A, Braiteh F S, Cassier P, DeLord J-P, Eder J P, Shen X, et al. Abstract PD1-6: Inhibition of PD-L1 by MPDL3280A leads to clinical activity in patients with metastatic triple-negative breast cancer. Cancer Res. 2015; 75(9 Supplement):PD1-6-PD1-6.
40. Bae S Y, La Choi Y, Kim S, Kim M, Kim J, Jung S P, et al. HER3 status by immunohistochemistry is correlated with poor prognosis in hormone receptor-negative breast cancer patients. Breast Cancer Res Treat. 2013; 139(3):741-50.
41. He T C, Zhou S, da Costa L T, Yu J, Kinzler K W, Vogelstein B. A simplified system for generating recombinant adenoviruses. Proc Natl Acad Sci USA. 1998; 95(5):2509-14.
42. Hodges B L, Serra D, Hu H, Begy C A, Chamberlain J S, Amalfitano A. Multiply deleted [E1, polymerase-, and pTP-] adenovirus vector persists despite deletion of the preterminal protein. The journal of gene medicine. 2000; 2(4):250-9.
43. Amalfitano A, Hauser M A, Hu H, Serra D, Begy C R, Chamberlain J S. Production and characterization of improved adenovirus vectors with the E1, E2b, and E3 genes deleted. J Virol. 1998; 72(2):926-33.
44. Rigby R A, Stasinopoulos D M. Generalized additive models for location, scale and shape. Journal of the Royal Statistical Society: Series C (Applied Statistics). 2005; 54(3):507-54.
45. Eilers P H C, Marx B D. Flexible smoothing with B-splines and penalties. 1996:89-121.
46. Hastie T, Tibshirani R. Varying-Coefficient Models. Journal of the Royal Statistical Society Series B (Methodological). 1993; 55(4):757-96.
47. Holm S. A Simple Sequentially Rejective Multiple Test Procedure. Scandinavian Journal of Statistics. 1979; 6(2):65-70.
48. van Buuren S, Fredriks M. Worm plot: a simple diagnostic device for modelling growth reference curves. Statistics in medicine. 2001; 20(8):1259-77.
49. DiCiccio T J, Efron B. Bootstrap confidence intervals. 1996:189-228.
50. Team RDC. R: A Language and Environment for Statistical Computing. the R Foundation for Statistical Computing. 2001.

1-7. (canceled)

8. A composition comprising:
a vaccine vector comprising a polynucleotide encoding a HER3 polypeptide, and
a checkpoint inhibitor or a polynucleotide encoding a checkpoint inhibitor.

9. The composition of claim 8, wherein the checkpoint inhibitor is selected from the group consisting of an anti-PD-1 agent, an anti-PDL1 agent, and an anti-CTLA-4 agent.

10. The composition of claim 8, wherein the composition comprises two checkpoint inhibitors, and wherein one checkpoint inhibitor comprises an anti-PD-1 agent or an anti-PDL1 agent and the other checkpoint inhibitor comprises an anti-CTLA-4 agent.

11. A pharmaceutical composition comprising a pharmaceutically-acceptable carrier and the composition of claim 8.

12. A method of treating a cancer or precancer or of reducing the likelihood of the cancer developing resistance to a cancer therapeutic or prevention agent in a subject comprising administering a therapeutically effective amount of the composition of claim 8 to the subject having the cancer or precancer.

13. A method of treating a cancer or precancer or of reducing the likelihood of the cancer developing resistance to a cancer therapeutic or prevention agent in a subject comprising:
administering a therapeutically effective amount of a vaccine vector comprising a polynucleotide encoding a HER3 polypeptide to the subject having the cancer or precancer, and
administering a therapeutically effective amount of a checkpoint inhibitor or a polynucleotide encoding a checkpoint inhibitor.

14. The method of claim 13, wherein the checkpoint inhibitor is selected from the group consisting of an anti-PD-1 agent, an anti-PDL1 agent, and an anti-CTLA-4 agent.

15. The method of claim 13, wherein two checkpoint inhibitors are administered, and wherein one checkpoint inhibitor comprises an anti-PD-1 agent or an anti-PDL1 agent and the other checkpoint inhibitor comprises an anti-CTLA-4 agent.

16. The method of claim 13, wherein the vaccine vector is administered prior to or simultaneously with the checkpoint inhibitor.

17. The method of claim 13, further comprising administering a therapeutically effective amount of the cancer therapeutic or prevention agent to the subject.

18. The method of claim 17, wherein the vaccine vector is administered prior to the administration of the cancer therapeutic or prevention agent.

19. The method of claim 13, wherein the cancer therapeutic or prevention agent is an agent targeting HER2, HER1, estrogen receptor, EGFR, or IGF1R.

20. The method of claim 13, wherein the cancer therapeutic or prevention agent is selected from the group consisting of trastuzumab, lapatinib, cetuximab, pertuzumab, and erlotanib.

21. The method of claim 13, wherein the cancer is HER2 or EGFR positive.

22. The method of claim 13, wherein the cancer or precancer is selected from a breast, prostate, lung, ovarian, colon, rectal, pancreas, gastric, bladder, head and neck, melanoma or liver cancer or precancer.

23. The method of claim 13, wherein administration of the vaccine vector and the checkpoint inhibitor results in decreased tumor growth rate or decreased tumor size after administration as compared to administration of either the vaccine vector or checkpoint inhibitor alone.
